(19) United States
(12) Patent Application Publication
(10) Pub. No.: US 2006/0281673 A1
(43) Pub. Date: Dec. 14, 2006

(54) Novel peptide inhibitor of hiv fusion that disrupts the internal trimeric coiled-coil of gp41

(76) Inventors: Marius Clore, Bethesda, MD (US); Carole A. Bewley-Clore, Bethesda, MD (US); John L. Medabalimi, Olney, MD (US)

Correspondence Address: BERENATO, WHITE & STAVISH, LLC, 6550 ROCK SPRING DR., SUITE 240, BETHESDA, MD, 20817, US

(21) Appl. No.: 10/544,115

(22) PCT Filed: Feb. 10, 2004
(86) PCT No.: PCT/US04/03794

§ 371 Date: Aug. 1, 2005

Related U.S. Application Data
(60) Provisional application No. 60/446,255, filed on Feb. 11, 2003.

(51) Int. Cl. A61K 38/17; C07K 14/16

(52) U.S. Cl. 514/12; 530/324

(57) ABSTRACT

This invention relates to a novel peptide inhibitor of a HIV fusion that disrupts the internal trimeric coiled-coil of gp41, to a pharmaceutical composition that comprise this inhibitor, and to methods of treating immunodeficiency disease, especially HIV, that employ such a pharmaceutical composition.

### CROSS-REFERENCE TO RELATED APPLICATIONS

This application claims priority to U.S. patent application Ser. No. 60/446,255, filed on Feb. 11, 2003, which application is herein incorporated by reference in its entirety.

### STATEMENT OF GOVERNMENTAL INTEREST

This invention was funded by the Laboratories of Bioorganic Chemistry, Chemical Physics, NIDDK, National Institutes of Health. This work was also supported by the Intramural AIDS Targeted Antiviral Program of the Office of the Director of the National Institutes of Health. The United States Government has certain rights to this invention.

### FIELD OF INVENTION

This invention relates to a novel peptide inhibitor of HIV fusion that disrupts the internal trimeric coiled-coil of gp41, to a pharmaceutical composition that comprise this inhibitor, and to methods of treating Immunodeficiency disease, especially HIV, that employ such a pharmaceutical composition.

### BACKGROUND OF THE INVENTION

The human immunodeficiency virus (HIV) is a pathogenic retrovirus (Varmus, H. (1988) “RETROVIRUSES,” Science 240:1427-1439; Cowley S. (2001) “THE BIOLOGY OF HIV INFECTION” Lepr Rev. 72(2):212-20). It is the causative agent of acquired immune deficiency syndrome (AIDS) and related disorders (Gallo, R. C. et al. (1983) “Isolation of human T-cell leukemia virus in acquired immune deficiency syndrome (AIDS),” Science 220(4599):865-7; Barre-Sinoussi, F. et al. “ISOLATION OF A T-LYMPHOTROPIC RETROVIRUS FROM A PATIENT AT RISK FOR ACQUIRED IMMUNE DEFICIENCY SYNDROME (AIDS),” (1983) Science 220:868-870; Gallo, R. et al. (1984) “FREQUENT DETECTION AND ISOLATION OF CYTOPATHIC RETROVIRUSES (HTLV-III) FROM PATENTS WITH AIDS AND AT RISK FOR AIDS,” Science 224:500-503; Teich, N. et al. (1984) “RNA TUMOR VIRUSES,” Weiss, R. et al. (eds.) Cold Spring Harbor Press (NY) pp. 949-956). HIV acts to compromise the immune system of infected individuals by targeting and infecting the CD-4⁺ T lymphocytes that would otherwise be the major proponents of the recipient's cellular immune system response (Dalgleish, A. et al. (1984) “THE CD4 (T4) ANTIGEN IS AN ESSENTIAL COMPONENT OF THE RECEPTOR FOR THE AIDS RETROVIRUS,” Nature 312: 767-768, Maddon et al. (1986) “THE T4 GENE ENCODES THE AIDS VIRUS RECEPTOR AND IS EXPRESSED IN THE IMMUNE SYSTEM AND THE BRAIN,” Cell 47:333-348, McDougal, J. S. et al. (1986) “BINDING OF HTLV-III/LAV To T4+ T CELLS BY A COMPLEX OF THE 110K VIRAL PROTEIN AND THE T4 MOLECULE,” Science 231:382-385). HIV infection is pandemic and HIV-associated diseases represent a major world health problem.

Attempts to treat HIV infection have focused on the development of drugs that disrupt the viral infection and replication cycle (see, Mitsuya, H. et al. (1991) “TARGETED THERAPY OF HUMAN IMMUNODEFICIENCY VIRUS-RELATED DISEASE,” FASEB J. 5:2369-2381). Such intervention could potentially inhibit the binding of HIV to cell membranes, the reverse transcription of the HIV RNA genome into DNA, the exit of the virus from the host cell and infection of new cellular targets, or inhibition of viral enzymes (see, U.S. Pat. No. 6,475,491). Thus, for example, soluble CD4 has been developed in an effort to competitively block the binding of HIV to lymphocytes (Smith, D. H. et al. (1987) “BLOCKING OF HIV-1 INFECTIVITY BY A SOLUBLE, SECRETED FORM OF THE CD4 ANTIGEN,” Science 238:1704-1707; Schooley, R. et al. (1990) “RECOMBINANT SOLUBLE CD4 THERAPY IN PATIENTS WITH THE ACQUIRED IMMUNODEFICIENCY SYNDROME (AIDS) AND AIDS-RELATED COMPLEX. A PHASE I-II ESCALATING DOSAGE TRIAL,” Ann. Int. Med. 112:247-253; Kahn, J. O. et al. (1990) “THE SAFETY AND PHARMACOKINETICS OF RECOMBINANT SOLUBLE CD4 (RCD4) IN SUBJECTS WITH THE ACQUIRED IMMUNODEFICIENCY SYNDROME (AIDS) AND AIDS-RELATED COMPLEX. A PHASE 1 STUDY,” Ann. Int. Med. 112:254-261, Yarchoan, R. et al. (1989) Proc. V^{th} Int. Conf. on AIDS, p564, MCP 137).

Likewise, drugs such as 2′,3′-dideoxynucleoside analogs (e.g., azidothymidine (AZT), dideoxyinosine, dideoxycytidine, and d4thymidine have been developed to target the virus' reverse-transcriptase (Yarchoan, R. et al. (1989) “CLINICAL PHARMACOLOGY OF 3′-AZIDO-2′,3′-DIDEOXYTHYMIDINE (ZIDOVUDINE) AND RELATED DIDEOXYNUCLEOSIDES,” N Engl J Med. 321(11):726-38). Recently, inhibitors of the HIV protease have been identified and used to treat HIV infection (see, U.S. Pat. No. 6,472,404; Todd, S. et al. (2000) “HIV PROTEASE AS A TARGET FOR RETROVIRUS VECTOR-MEDIATED GENE THERAPY,” Biochim Biophys Acta. 1477(1-2): 168-88).

HIV infection is believed to occur through the fusion of viral-cell and cell-cell membranes. This process is mediated by the gp41 and gp120 HIV env proteins and the cellular CD4 protein. Following binding of gp120 to CD4, a conformational change occurs in the gp120/gp41 complex. This change leads to the insertion of the gp41 protein into the target membrane and ultimately to membrane fusion. Agents that interfere with hairpin formation can inhibit HIV-1 infection (Root, M. J. et al. (2001) “PROTEIN DESIGN OF AN HIV-1 ENTRY INHIBITOR” Science 291 884-888; Kilby, J. M. et al. (1998) “POTENT SUPPRESSION OF HIV-1 REPLICATION IN HUMANS BY T-20, A PEPTIDE INHIBITOR OF GP41-MEDIATED VIRUS ENTRY” Nature Medicine 4(11) 1302-1307). Thus, various domains of gp41 have been implicated as possible inhibitors of HIV infection (Wild, C. T. et al. (1994) “PEPTIDES CORRESPONDING TO A PREDICTIVE α-HELICAL DOMAIN OF HUMAN IMMUNODEFICIENCY VIRUS TYPE GP41 ARE POTENT INHIBITORS OF VIRUS INFECTION,” Proc. Natl. Acad. Sci. (U.S.A.) 91 9770-9774; Wild, C. T. et al. (1992) “A SYNTHETIC INHIBITOR OF HUMAN IMMUNODEFICIENCY VIRUS REPLICATION: CORRELATION BETWEEN SOLUTION STRUCTURE AND VIRAL INHIBITION,” Proc. Natl. Acad. Sci. (U.S.A.) 89 10537-10541).

The gp41 region containing the naturally occurring sequence of residues 546-581 of the HIV-1 Env protein has been previously found to possess HIV-inhibitory activity (Louis, J. M. et al. (2001) “DESIGN AND PROPERTIES OF N_{CCG}-GP41, A CHIMERIC GP41 MOLECULE WITH NANOMOLAR HIV FUSION INHIBITORY ACTIVITY” J. Biol. Chem. 276 (31, Issue of Aug. 3, 2001) 29485-29489). In addition, researchers have explored the effects of mutation in this region on membrane fusion (Weng, Y. et al. (1998) “MUTATIONAL ANALYSIS OF RESIDUES IN THE COILED-COIL DOMAIN OF HUMAN IMMUNODEFICIENCY VIRUS TYPE 1 TRANSMEMBRANE PROTEIN GP41” J. Virol. 72(12) 9676-82).

Significantly, despite substantial research into the causes and treatment of HIV (see, for example, US20020106374a1; WO0232452a1; WO0224735a2; WO0222077a3; WO0222077a2; U.S. Pat. No. 6,333,395; U.S. Pat. No. 6,331,404;US20010047080a1; U.S. Pat. No. 6,271,198; WO0155439a1; EP0538283b2; WO0151673a2; WO0151673a1; U.S. Pat. No. 6,258,782; WO0144286a3; WO0144286a2; WO0137881a3; WO0137881a2; U.S. Pat. No. 6,228,983; EP0572737b1; U.S. Pat. No. 6,171,596; U.S. Pat. No. 6,150,088; WO0055377a1; U.S. Pat. No. 6,093,794; EP1019511a2; U.S. Pat. No. 6,068,973; WO0006599a1; U.S. Pat. No. 6,020,459; U.S. Pat. No. 6,017,536; U.S. Pat. No. 6,013,263; EP0652895b1; U.S. Pat. No. 5,864,027; U.S. Pat. No. 5,834,267; WO9820036a1; U.S. Pat. No. 5,736,391; EP0793675a1; EP774971a1; EP0538283b1; U.S. Pat. No. 5,576,421; WO9619495a1; EP0674657a1; EP0572737a3; WO9412533a1; EP0572737a2; EP0538283a1; WO9200997a1; and WO8909785a1), suitable inhibitors of HIV fusion, that possess the ability to disrupt the internal trimeric coiled-coil of gp41 have not been identified The present invention is directed to such inhibitors.

### SUMMARY OF THE INVENTION

The pre-hairpin intermediate of gp41 from the human immunodeficiency virus (HIV) is the target for two classes of fusion inhibitors that bind to the C-terminal region or the trimeric coiled-coil of N-terminal helices, thereby preventing formation of the fusogenic trimer of hairpins. Using rational design, two 36-residue peptides, N36^{Mut(e,g)} and N36^{Mut(a,d)} were derived from the parent N36 peptide comprising the N-terminal helix of the gp41 ectodomain (residues 546-581 of HIV-1 envelope), characterized by analytical ultracentrifugation and CD, and assessed for their ability to inhibit HIV fusion using a quantitative vaccinia virus-based fusion assay. N36^{Mut(e,g)} contains nine amino acid substitutions designed to disrupt interactions with the C-terminal region of gp41 while preserving contacts governing the formation of the trimeric coiled-coil. N36^{Mut(a,d)} contains nine substitutions designed to block formation of the trimeric coiled-coil but retains residues that interact with the C-terminal region of gp41. N36^{Mut(a,d)} is monomeric, is largely random coil, does not interact with the C34 peptide derived from the C-terminal region of gp41 (residues 628-661), and does not inhibit fusion. The trimeric coiled-coil structure is therefore a prerequisite for interaction with the C-terminal region of gp41. N36^{Mut(e,g)} forms a monodisperse, helical trimer in solution, does not interact with C34, and yet inhibits fusion about 50-fold more effectively than the parent N36 peptide (IC₅₀˜308 nM versus˜16 μM). These results indicate that N36^{Mut(e,g)} acts by disrupting the homotrimeric coiled-coil of N-terminal helices in the pre-hairpin intermediate to form heterotrimers. Thus N36^{Mut(e,g)} represents a novel third class of gp41-targeted HIV fusion inhibitor. A quantitative model describing the interaction of N36^{Mut(e,g)} with the pre-hairpin intermediate is presented.

This invention thus relates to a novel peptide inhibitor of HIV fusion that disrupts the internal trimeric coiled-coil of gp41, to a pharmaceutical composition that comprise this inhibitor, and to methods of treating Immunodeficiency disease, especially HIV, that employ such a pharmaceutical composition.

The invention is more fully described in Bewley, C. A., Louis, J. M., Ghirlando, R. and Clore, G. M. (2002) “DESIGN OF A NOVEL PEPTIDE INHIBITOR OF HIV FUSION THAT DISRUPTS THE INTERNAL TRIMERIC COILED-COIL OF GP41,” J. Biol. Chem. 277 (16, Issue of Apr. 19, 2002) 14238-14245), herein incorporated by reference in its entirety. An on-line version of this publication was published on Feb. 21, 2002 (M201453200); see also, Sanders, R. W. et al. (Jul. 29, 2002) “STABILIZATION OF THE SOLUBLE, CLEAVED, TRIMERIC FORM OF THE ENVELOPE GLYCOPROTEIN COMPLEX OF HUMAN IMMUNODEFICIENCY VIRUS TYPE 1,” J. Virol. 76:8875-8889; Follis, K. E. et al. (Jun. 14, 2002). “GENETIC EVIDENCE THAT INTERHELICAL PACKING INTERACTIONS IN THE GP41 CORE ARE CRITICAL FOR TRANSITION OF THE HUMAN IMMUNODEFICIENCY VIRUS TYPE 1 ENVELOPE GLYCOPROTEIN TO THE FUSION-ACTIVE STATE,” J. Virol. 76:7356-7362.

The invention further concerns the use of such peptides in the treatment of HIV transmission, and in the treatment of AIDS. The invention additionally concerns a method of attenuating the transmission or infection of HIV into cells, and of treating AIDS, comprising providing one or more of such peptide(s) to such cells.

In detail, the invention concerns a compound, comprising an N36 peptide variant or a derivative or pharmaceutically acceptable salt thereof, wherein the compound inhibits the fusion of HIV-1 to a human cell.

The invention particularly concerns the embodiments of such compounds wherein the identities of the amino acid residues occupying the “e” and “g” positions of the N36 peptide variant are at most 89% identical to the amino acid residues occupying the “e” and “g” positions of N36, and the identities of the amino acid residues occupying the “non-e” and “non-g” positions of the N36 peptide variant are at least 70% identical to the amino acid residues occupying their counterpart positions in the N36 peptide; wherein the N36 peptide variants can no longer significantly interact with the C-region of gp41 and therefore possess the ability to disrupt the internal trimeric coiled-coil of N-helices of gp41.

The invention further particularly concerns the embodiments of such compounds wherein the N36 peptide variant differs in amino acid sequence from the sequence of N36 peptide (SEQ ID NO:1), by at least a substitution of an amino acid residue, two, three, four, five, six, seven, eight, or nine amino acid residues selected from the group consisting of:: V₄, Q₆, L₁₁, A₁₃, Q₁₈, L₂₀, V₂₅, G₂₇ and Q₃₂, wherein the letters V, Q, L, A and G denote Valine, Glutamine, Leucine, Alanine and Glycine, respectively, and the number denotes the position of the residue in SEQ ID NO:1.

The invention particularly concerns the embodiment of such compounds wherein the N36 peptide variant has the amino acid sequence of N36^{Mut(e,g)} (SEQ ID NO:3):

SGIDQEQNNL TRLIEAQIHE LQLTQWKIKQ LLARIL.

The invention additionally provides a pharmaceutical composition comprising a therapeutically effective amount of an N36 peptide variant or a derivative or pharmaceutically acceptable salt thereof, wherein the compound inhibits the fusion of HIV-1 to a human cell, in admixture with a pharmaceutically acceptable excipient.

The invention particularly concerns the embodiments of such pharmaceutical compositions wherein the identities of the amino acid residues occupying the “e” and “g” positions of the N36 peptide variant are at most 89% identical to the amino acid residues occupying the “e” and “g” positions of N36, and the identities of the amino acid residues occupying the “non-e” and “non-g” positions of the N36 peptide variant are at least 70% identical to the amino acid residues occupying their counterpart positions in the N36 peptide; wherein the N36 peptide variants can no longer significantly interact with the C-region of gp41 and therefore possess the ability to disrupt the internal trimeric coiled-coil of N-helices of gp41.

The invention further particularly concerns the embodiments of such pharmaceutical compositions wherein the N36 peptide variant differs in amino acid sequence from the sequence of N36 peptide (SEQ ID NO:1), by at least a substitution of an amino acid residue, two, three, four, five, six, seven, eight, or nine amino acid residues selected from the group consisting of: V₄, Q₆, L₁₁, A₁₃, Q₁₈, L₂₀, V₂₅, G₂₇ and Q₃₂, wherein the letters V, Q, L, A and G denote Valine, Glutamine; Leucine, Alanine and Glycine, respectively, and the number denotes the position of the residue in SEQ ID NO:1.

The invention particularly concerns the embodiment of such pharmaceutical compositions wherein the N36 peptide variant has the amino acid sequence of N36^{Mut(e,g)} (SEQ ID NO:3):

SGIDQEQNNL TRLIEAQIHE LQLTQWKIKQ LLARIL.

The invention particularly concerns the embodiment of all such pharmaceutical compositions, wherein the composition additionally contains an HIV protease inhibitor, an HIV reverse transcriptase inhibitor, an HIV integrase inhibitor, or an HIV fusion inhibitor.

The invention additionally provides a method of treating HIV infection that comprises providing to a recipient a therapeutically effective or a prophylactically effective amount of a pharmaceutical composition comprising a therapeutically effective amount of an N36 peptide variant or a derivative or pharmaceutically acceptable salt thereof, wherein the compound inhibits the fusion of HIV-1 to a human cell, in admixture with a pharmaceutically acceptable excipient.

The invention particularly concerns the embodiments of such method wherein the identities of the amino acid residues occupying the “e” and “g” positions of the N36 peptide variant are at most 89% identical to the amino acid residues occupying the “e” and “g” positions of N36, and the identities of the amino acid residues occupying the “non-e” and “non-g” positions of the N36 peptide variant are at least 70% identical to the amino acid residues occupying their counterpart positions in the N36 peptide; wherein the N36 peptide variants can no longer significantly interact with the C-region of gp41 and therefore possess the ability to disrupt the internal trimeric coiled-oil of N-helices of gp41.

The invention further particularly concerns the embodiments of all such methods wherein the N36 peptide variant differs in amino acid sequence from the sequence of N36 peptide (SEQ ID NO:1), by at least a substitution of an amino acid residue, two, three, four, five, six, seven, eight, or nine amino acid residues selected from the group consisting of: V₄, Q₆, L₁₁, A₁₃, Q₁₈, L₂₀, V₂₅, G₂₇ and Q₃₂, wherein the letters V, Q, L, A and G denote Valine, Glutamine, Leucine, Alanine and Glycine, respectively, and the number denotes the position of the residue in SEQ ID NO:1.

The invention particularly concerns the embodiments of all such methods wherein the N36 peptide variant has the amino acid sequence of N36^{Mut(e,g)} (SEQ ID NO:3):

SGIDQEQNNL TRLIEAQIHE LQLTQWKIKQ LLARIL.

The invention particularly concerns the embodiments of all such methods wherein the composition additionally contains an HIV protease inhibitor, an HIV reverse transcriptase inhibitor, an HIV integrase inhibitor, or an HIV fusion inhibitor

The invention particularly concerns the use of the above compounds and pharmaceutical compositions to treat HIV infection in humans, FIV infection in felines, and SIV infection in simians.

### BRIEF DESCRIPTION OF THE FIGURES

FIG. 1 shows a schematic model illustrating the site of action of different HIV fusion inhibitors that target the ectodomain of gp41. Panel a, the fusogenic state of gp41 (bottom) consists of a trimer of hairpins comprising an internal trimeric, helical coiled-coil of the N-region surrounded by helices derived from the C-region (Caffrey, M. et al. (1998) “THREE-DIMENSIONAL SOLUTION STRUCTURE OF THE 44 KDA ECTODOMAIN OF SIV GP41,” EMBO J. 17:4572-4584; Chan, D. C. et al. (1997) “CORE STRUCTURE OF GP41 FROM THE HIV ENVELOPE GLYCOPROTEIN,” Cell 89:263-273; Weissenhorn, W. et al. (1997) “ATOMIC STRUCTURE OF THE ECTODOMAIN FROM HIV-1 GP41,” Nature 387:426-430; Tan, K. J. et al. (1997) “ATOMIC STRUCTURE OF A THERMOSTABLE SUBDOMAIN OF HIV-1 GP41,” Proc. Natl. Acad. Sci. (U.S.A.) 94:12303-12308; Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139). The inhibitors target a pre-hairpin intermediate state (top) in which the N- and C- regions of gp41 are not yet associated (Eckert, D. M. et al. (2001) “MECHANISMS OF VIRAL MEMBRANE FUSION AND ITS INHIBITION,” Annu. Rev. Biochem. 70:777-810). In the pre-hairpin intermediate state, the N-region is thought to consist of a trimeric, parallel helical coiled-coil; the fusion peptide (green) located at the N terminus of the ectodomain of gp41 is inserted into the target cell membrane; the C-region of gp41 is anchored to the viral membrane by a transmembrane segment (purple). Peptides derived from the C-region, such as C34 (Chan, D. C. et al (1998) “EVIDENCE THAT A PROMINENT CAVITY IN THE COILED COIL OF HIV TYPE 1 GP41 IS AN ATTRACTIVE DRUG TARGET,” Proc. Natl. Acad. Sci. U.S.A. 95:15613-15617), bind to the N-region in its trimeric coiled-coil state; the proteins N_{CCG}-gp41 (Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489) and 5-helix (Root, M. J. et al. (2001) “PROTEIN DESIGN OF AN HIV-1 ENTRY INHIBITOR,” Science 291:884-888), which expose either the complete or a portion of the N-region trimeric coil-coil in a stable form, bind to the C-region. N36^{Mut(e,g)}, the subject of the present article, has been designed to remove the interaction surface between the N- and C-regions and therefore can only interact with the N-region in a monomeric form, thereby disrupting the homotrimeric coiled-coil N-region and resulting in the formation of heterotrimers. In all three instances, the fusion inhibitors block the formation of the trimer of hairpins, thereby preventing apposition of the viral and target cell membranes. Panel b, as a consequence of the existence of a monomer-trimer equilibrium for the trimeric coiled-coil of N-helices, the interaction of homotrimeric N36^{Mut(e,g)} (yellow) with the fusion-competent homotrimeric pre-hairpin intermediate (N-helices) results in subunit exchange and the formation of fusion-incompetent heterotrimers.

FIG. 2 shows the design of a peptide that disrupts the internal N-region trimeric coiled-coil in the pre-hairpin intermediate state of gp41. Panel a, helical wheel representation illustrating the interaction between the N- and C-regions of gp41 in the trimer of hairpins as observed in the solution (Caffrey, M. et al. (1998) “THREE-DIMENSIONAL SOLUTION STRUCTURE OF THE 44 KDA ECTODOMAIN OF SIV GP41,” EMBO J. 17:4572-4584) and crystal (Chan, D. C. et al. (1997) “CORE STRUCTURE OF GP41 FROM THE HIV ENVELOPE GLYCOPROTEIN,” Cell 89:263-273; Weissenhorn, W. et al. (1997) “ATOMIC STRUCTURE OF THE ECTODOMAIN FROM HIV-1 GP41,” Nature 387:426-430; Tan, K. J. et al. (1997) “ATOMIC STRUCTURE OF A THERMOSTABLE-SUBDOMAIN OF HIV-1 GP41,” Proc. Natl. Acad. Sci. (U.S.A.) 94:12303-12308; Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139) structures of the fusogenic/postfusogenic state of the ectodomain of gp41. The intermolecular contacts between the N-helices occur between positions a and d of the helical wheel. Contacts between the N- and C-helices (intra- and intermolecular) involves residues at positions e and g of the N-helices and positions a and d of the C-helices. Panel b, peptide sequences. The N36 peptide comprises residues 546-581 of the N-region of HIV-1gp41, and the C34 peptide comprises residues 628-661 of the C-region of HIV-1 gp41. N36 and C34 associate to form a six-helix bundle whose structure has been solved crystallographically (Chan, D. C. et al. (1997) “CORE STRUCTURE OF GP41 FROM THE HIV ENVELOPE GLYCOPROTEIN,” Cell 89:263-273). In the N36^{Mut(e,g)} mutant, the residues at positions e and g of N36 have been substituted by residues at positions e and g, respectively, of C34; this effectively removes the interaction surface with C34 but preserves the contacts necessary to form a trimeric coiled-coil of N-helices. In the N36^{Mut(a,d)} mutant, the residues at positions a and d of N36 have been substituted by residues at positions f and c, respectively, of C34; this removes the contacts necessary to form the trimeric coil-coil of N-helices but preserves the interaction sites with C34.

FIG. 3 shows the characterization of N36^{Mut(e,g)} and N36^{Mut(a,d)}. Panel a, results of analytical ultracentrifugation on N36^{Mut(e,g)} (top) and N36^{Mut(a,d)} (center). Sedimentation equilibrium profiles, plotted in terms of ln(A₂₈₀) versus the square of the radius (r²) (bottom panel), are shown; also shown in the top two panels is the distribution of residuals between calculated and experimental data for best fits to a monomer of N36^{Mut(a,d)} and a trimer of N36^{Mut(e,g)}. The concentrations (in monomer) of N36^{Mut(a,d)} and N36^{Mut(e,g)} are ˜1.40 μM (A₂₈₀˜0.8) and 124 μm (A₂₈₀˜0.7), respectively. The calculated molecular masses are 3660±80 Da for N36^{Mut(a,d)}, which corresponds to a monomer, and 12,040±200 Da for N36^{Mut(e,g)}, which corresponds to a trimer. An independent run for N36^{Mut(e,g)} at a concentration of 36 μM in monomer (A₂₈₀˜0.2) yielded a molecular mass of 12,500˜180 Da. b, CD spectrum of N36^{Mut(e,g)} and N36^{Mut(a,d)}. The calculated helical content is ˜80% for N36^{Mut(e,g)} and ˜20% for N36^{Mut(a,d)} deg, degrees. Panel b, the mean residue ellipticity plotted as a function of wavelength for N36^{Mut(e,g)} and N36^{Mut(a,d)}.

FIG. 4 shows the inhibition of HIV Env-mediated cell fusion by N36^{Mut(e,g)}, N36^{Mut(a,d)}, and N36 circles, N36^{Mut(e,g)}, solid squares, N36^{Mut(a,d)}; black open circles, N36. The solid lines represent best fits to the data using the simple activity relationship: % fusion 100/(1+[I]/IC₅₀) where [I] is the inhibitor concentration. The IC₅₀ values for N36^{Mut(e,g)} and N36 are 308±22 nM and 16±2 μM, respectively. N36^{Mut(a,d)} displays no inhibitory activity at the concentrations tested.

FIG. 5 models the inhibition of HIV Env-mediated cell fusion by N36^{Mut(e,g)}. Panel a, mechanistic scheme. L, LL, and LLL are the monomeric, homodimeric, and homotrimeric forms, respectively, of the ligand N36^{Mut(eg,)}; M, MM, and MMM are the monomeric, homodimeric, and homotrimeric forms, respectively, of the prefusion intermediate of gp41 bound on the surface of the cell; ML is the heterodimeric species formed by the interaction of M and L; MML and MLL are the heterotrimeric species. K_{ref}^{trimer} is the experimentally measured equilibrium association constant for the monomer-trimer equilibrium of the ectodomain of gp41 in free solution (4.8×10¹¹ M⁻²; Wingfield, P. T. et al. (1997) “THE EXTRACELLULAR DOMAIN OF IMMUNODEFICIENCY VIRUS GP41 PROTEIN: EXPRESSION IN ESCHERICHIA COLI, PURIFICATION, AND CRYSTALLIZATION,” Protein Sci. 6, 1653-1660) given by the product of K₁ and K₂ with K₂>>K₁ (since trimer formation is highly cooperative, and only monomer and trimer species can be detected by analytical ultracentrifugation). (K₁ was arbitrarily set to 10⁴ M⁻¹, yielding a value of 4.8×10⁷ M⁻¹ for K₂.) The factors α, β and λ relate the equilibrium association constants for homotrimerization of L (K_{homo,L}^{trimer}), heterotrimerization of M and L (K_{hetero,ML}^{trimer}); and homodimerization of M (K_{homo,M}^{trimer,spp}) to K_{ref}^{trimer}. The factor λ serves to convert the concentrations of species in the membrane to their bulk solution concentrations and, in addition, subsumes any energetic differences between trimerization of the pre-hairpin intermediate of gp41 in the membrane and trimerization of the ectodomain of gp41 measured in free solution. The various numerical factors in front of the equilibrium constants are statistical factors related to symmetry considerations involved in the formation of homo- and hetero-oligomeric species. Panel b, variation in the optimized values of α and β derived by nonlinear least-squares optimization as a function of λM_{T}, where M_{T} is the total concentration of protein (monomer units) in bulk solution. The vertical bars represent the error in the fitted parameters. Panel c, comparison of the experimental fusion data (solid circles) with the best fit theoretical curves calculated for λM_{T}=1.5×10⁻⁵ M (solid line) and 1.5×10⁻⁴ M (dashed line). (For a value of 10 pM for M_{T}, the corresponding values of λ are 1.5×10⁶−1.5×10⁷, respectively). The percentage of fusion activity is given by 100[MMM]_{LT}/[MMM]_{LT=0}. Note that the two theoretical curves are essentially indistinguishable not only from each other but also from a simple Langmuir isotherm. Calculated fractional concentrations of various species as a function of total protein concentration, M_{T} (Panel d), and total ligand concentration, L_{T} (Panel e). The fraction of dimeric species (i.e. 2[MM]/M_{T}, [ML]/M_{T}, and 2[LL]/L_{T}) is less than 1% for all values of L_{T}. The curves obtained for λM_{T}=1.5×10⁻⁵ M and 1.5×10⁻⁴ M are shown as solid and dashed lines, respectively.

### DESCRIPTION OF THE PREFERRED EMBODIMENTS

The abbreviations used herein are: Env, viral envelope glycoprotein(s); HIV, human immunodeficiency virus; gp120, surface envelope glycoprotein of HIV; gp41, transmembrane subunit of HIV envelope; N36 and C34, peptides encompassing residues 546-581 and 628-661 of HIV-1 Env, respectively; N36^{Mut(e,g)}, peptide derived from N36 that contains nine substitutions at positions e and g of the helical wheel (defined in the context of the gp41 trimer of hairpins structure) corresponding to residues 549, 551, 556, 558, 563, 565, 570, 572, and 577 of HIV-1 Env; N36^{Mut(a,d)}, peptide derived from N36 that contains nine substitutions at positions a and d of the helical wheel (defined in the context of the gp41 trimer of hairpins structure) corresponding to residues 552, 555, 559, 562, 566, 569, 573, 576, and 580 of HIV-1 Env.

As used herein, the “N36 peptide” has the amino
acid sequence:

(SEQ ID NO:1)

SGIVQQQNNL LRAIEAQQHL LQLTVWGIKQ LQARIL
1 10 20 30 36
As used herein, the “C34 peptide” has the amino
acid sequence:

(SEQ ID NO:2)

WMEWDREINN YTSLIHSLIE EESQNQQEKN EQELL
1 10 20 30 35

The invention particularly concerns peptide variants that are variants of the N36 peptide, as well as peptide derivatives thereof (e.g., salts, peptide conjugates, etc.), that increase the stability of the peptide-gp41 heterotrimer over the peptide and gp41 homotrimers, and which therefore can be used to inhibit HIV-1 fusion. As used herein, a “peptide variant” of another peptide is a peptide molecule (or derivatized peptide) whose amino acid sequence differs from such other peptide, but is at least 95%, at least 90%, at least 80%, at least 75%, at least 70%, at least 65%, at least 60%, or at least 50%, identical to such other sequence. The invention particularly concerns such peptide variants of the N36 peptide that can no longer significantly interact with the C-region of gp41 and therefore possess the ability to disrupt the internal trimeric coiled-coil of N-helices of gp41. As used herein, a peptide can no longer “significantly” interact with the C-region of gp41 if it is capable of inhibiting HIV-1 fusion. As used herein, a peptide is said to inhibit HIV-1 fusion, if, relative to the fusion observed in the presence of N36 peptide fusion in the presence of such peptide is inhibited 2-fold, 5-fold, 10-fold, 15-fold, 20-fold; 25-fold, 30-fold, 35-fold, 40-fold, 45-fold, 50-fold, or more.

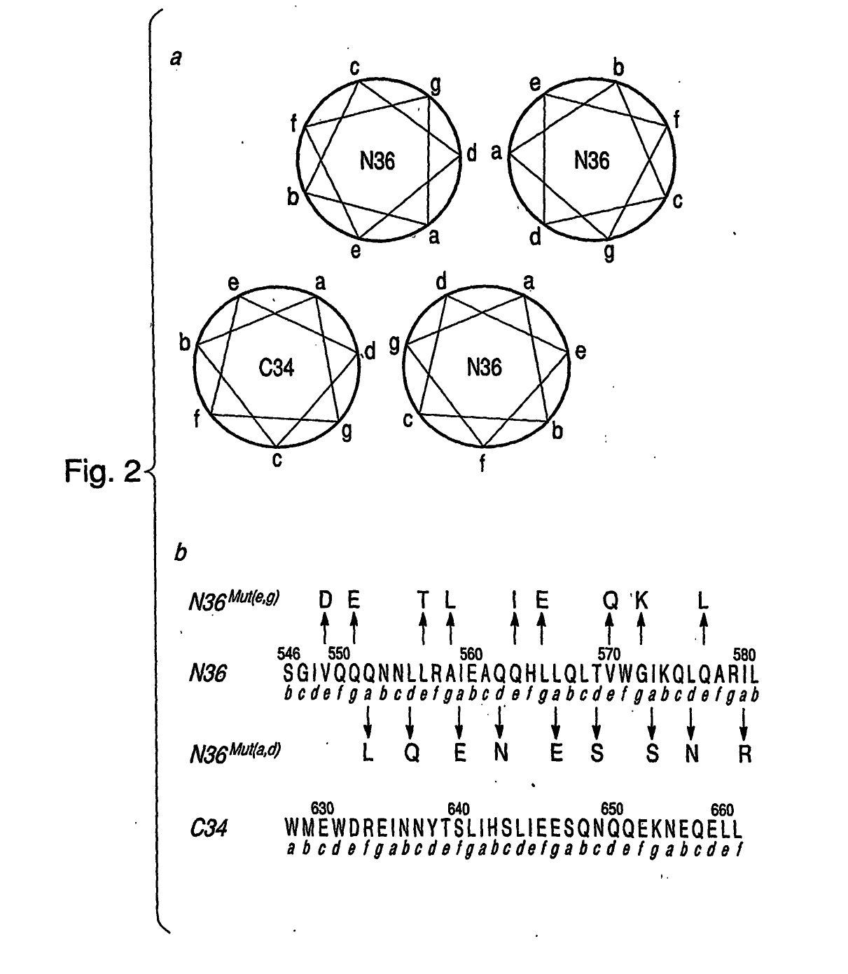

The invention particularly concerns such peptide variants of the N36 peptide in which the identities of one or more of the amino acid residues occupying the “e” and “g” positions of the N36 peptide variant differ from those of the N36 peptide (see, FIG. 2, Panel b). As used herein, an “e” residue of the N36 peptide is an amino acid residue occupying position 4, 11, 18, 25 and/or 32 of the N36 peptide. Thus, the “e” residues of the N36 peptide are selected from the group consisting of N36 residues: V₄, L₁₁, Q₁₈, V₂₅, and Q₃₂ (wherein the letters V, L and Q denote Valine, Leucine, and Glutamine, respectively, and the number denotes the position of the residue in SEQ ID NO:1). As used herein, a “g” residue of the N36 peptide is an amino acid residue occupying position 6, 13, 20, and/or 27 of the N36 peptide. Thus, the “g” residues of the N36 peptide are selected from the group consisting of N36 residues: Q₆, A₁₃, L₂₀, and G₂₇ (wherein the letters Q, A, L, and G denote Glutamine, Alanine, Leucine, and Glycine, respectively, and the number denotes the position of the residue in SEQ ID NO:1).

As used herein, a “non-e” and “non-g” residue of the N36 peptide is an amino acid residue occupying positions 1-3, 5, 7-10, 12, 14-17, 19, 21-24, 26, 28-31, 33-36 of N36 (see, FIG. 2, Panel b). Thus, the “non-e” and “non-g” residues of the N36 peptide are residues S₁, G₂, I₃, Q₅, Q₇, N₈, N₉, L₁₀, R₁₂, I₁₄, E₁₅, A₁₆, Q₁₇, H₁₉, L₂₁, Q₂₂, L₂₃, T₂₄, W₂₆, I₂₈, K₂₉, Q₃₀, L₃₁, A₃₃, R₃₄, I₃₅, and L₃₆ of SEQ ID NO:1, wherein the letters S, G, I, Q, N, L, R, E, A, H, T, W, and K denote Serine, Glycine, Glutamine, Isoleucine, Asparagines, Leucine, Arginine, Glutamate, Alanine, Histidine, Threonine, Tryptophan, and Lysine, respectively, and the number denotes the position of the residue in SEQ ID NO:1. The identities of the amino acid residues occupying the “non-e” and “non-g” positions of the N36 peptide variants of the present invention may be the same or different (but are preferably at least 70% identical) from their counterparts in the N36 peptide.

In preferred embodiments, the peptides variants of the N36 peptide will thus differ in their amino acid sequence from the sequence of SEQ ID NO:1, by the substitution of a single “e” or “g” amino acid residue (97.2% identical to SEQ ID NO:1), more preferably by the substitution of two such amino acid residues (94.4% identical to SEQ ID NO:1), still more preferably by the substitution of three such amino acid residues (91.7% identical to SEQ ID NO:1), still more preferably by the substitution of four such amino acid residues (88.9% identical to SEQ ID NO:1), still more preferably by the substitution of five such amino acid residues (86.1% identical to SEQ ID NO:1), still more preferably by the substitution of six such amino acid residues (83.3% identical to SEQ ID NO:1), still more preferably by the substitution of seven such amino acid residues (80.6% identical to SEQ ID NO:1), still more preferably by the substitution of eight such amino acid residues (77.8% identical to SEQ ID NO:1), or still more preferably by the substitution of nine such amino acid residues (75% identical to SEQ ID NO:1). The 1-9 amino acid residues that are preferably substituted for of N36 residues: V₄, Q₆, L₁₁, A₁₃, Q₁₈, L₂₀, V₂₅, G₂₇ or Q₃₂ may be selected from any naturally occurring or modified amino acid residue. Preferably, however, since the purpose of the substitution is to diminish or eliminate the contact(s) between an “e” or “g” residue of the N36 peptide variant that are involved in the interaction of the N36 peptide with the C-region of gp41, substitutions that are non conservative (i.e., polar for non-polar, positively charged for negatively charged, bulky side groups for compact side groups, etc.) are preferred. In such N36 peptide variants, “non-e” and/or “non-g” N36 amino acid residues can be replaced with naturally occurring or modified amino acid residue(s), provided that at least 75% sequence identity (with respect to the “non-e” and/or “non-g” amino acid residues of N36) remain.

In a particularly preferred such sub-embodiment, the peptide variant of the present invention will be the N36^{Mut(e,g)} peptide, having the amino acid sequence:

(SEQ ID NO:3)

SGIDQEQNNL TRLIEAQIHE LQLTQWKIKQ LLARIL
1 10 20 30 36

as well as mutated forms of this sequence that yield peptides that increase the stability of the peptide-gp41 heterotrimer over the peptide and gp41 homotrimers. Particularly preferred mutated forms of this sequence are those that possess single, double, etc. conservative point mutations at positions a or d of the helical wheel representation of this sequence (see FIG. 2, Panel a). Methods of making such mutated forms of an amino acid sequence are well known in the art.

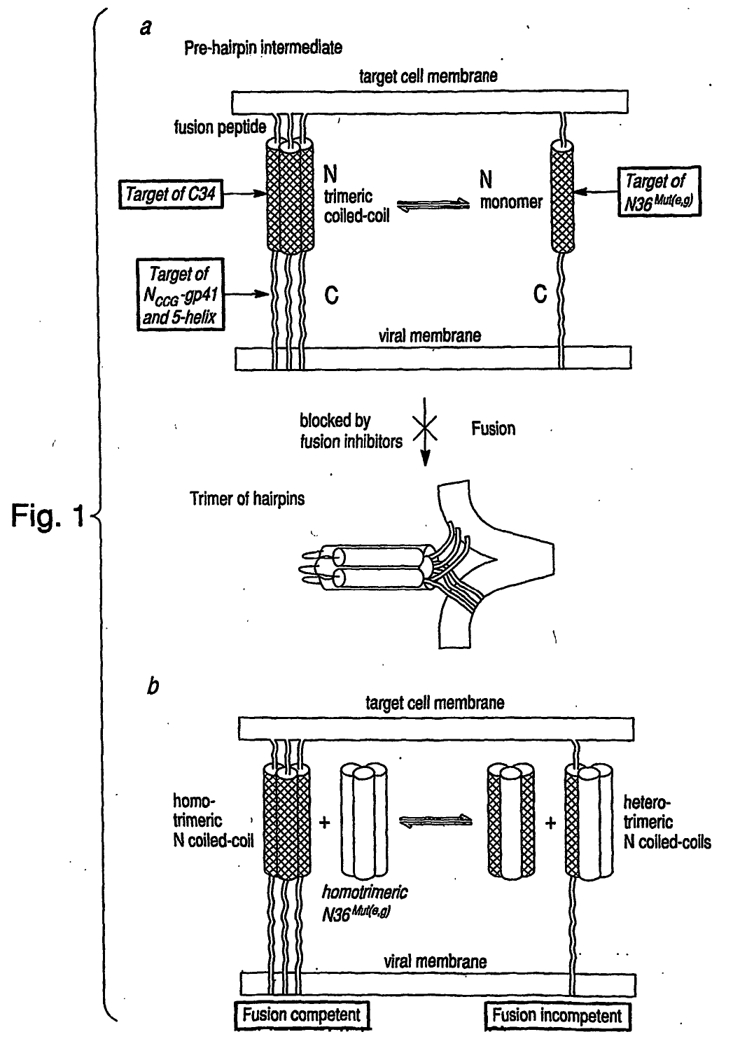

Virus-cell and cell-cell fusion mediated by the viral envelope glycoproteins (Env) (Freed, E. O. et al. (1995) “THE ROLE OF HUMAN IMMUNODEFICIENCY VIRUS TYPE 1 ENVELOPE GLYCOPROTEINS IN VIRUS INFECTION,” J. Biol. Chem. 270:23883-23886) gp41 and gp120 constitute the first step of infection and dissemination, respectively, of the human immunodeficiency virus (HIV) and hence represent a promising target for the development of antiviral therapeutics (Eckert, D. M. et al. (2001) “MECHANISMS OF VIRAL MEMBRANE FUSION AND ITS INHIBITION,” Annu. Rev. Biochem. 70:777-810). Following binding of gp120 to CD4 and a chemokine receptor, a conformational change occurs in the gp120/gp41 oligomer that leads to insertion of the fusion peptide of gp41 into the target membrane and ultimately membrane fusion (Eckert, D. M. et al. (2001) “MECHANISMS OF VIRAL MEMBRANE FUSION AND ITS INHIBITION,” Annu. Rev. Biochem. 70:777-810; Moore, J. P. et al. (1997) “CO-RECEPTORS FOR HIV-1 ENTRY,” Curr. Opin. Immunol. 9:551-562). The structure of the ectodomain of both HIV and simian immunodeficiency virus gp41 in its fusogenic/postfusogenic state has been solved by NMR (Caffrey, M. et al. (1998) “THREE-DIMENSIONAL SOLUTION STRUCTURE OF THE 44 KDA ECTODOMAIN OF SIV GP41,” EMBO J. 17:4572-4584) and crystallography (Chan, D. C. et al. (1997) “CORE STRUCTURE OF GP41 FROM THE HIV ENVELOPE GLYCOPROTEIN,” Cell 89:263-273; Weissenhorn, W. et al. (1997) “ATOMIC STRUCTURE OF THE ECTODOMAIN FROM HIV-1 GP41,” Nature 387:426-430; Tan, K. J. et al. (1997) “ATOMIC STRUCTURE OF A THERMOSTABLE SUBDOMAIN OF HIV-1 GP41,” Proc. Natl. Acad. Sci. (U.S.A.) 94:12303-12308; Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139) and shown to consist of a trimer of hairpins. Each subunit comprises long N- and C-terminal helices connected by a 25-30-residue loop. The N-helices form a parallel, trimeric coiled-coil in the interior of the complex surrounded by the C-terminal helices oriented antiparallel to the N-terminal helices (FIG. 1, Panel a, bottom). Peptides derived from the C- and N-helices inhibit Env-mediated fusion at nanomolar and micromolar concentrations, respectively (Wild, C. T., et al. (1992) “A SYNTHETIC PEPTIDE INHIBITOR OF HUMAN IMMUNODEFICIENCY VIRUS REPLICATION: CORRELATION BETWEEN SOLUTION STRUCTURE AND VIRAL INHIBITION,” Proc. Natl. Acad. Sci. U.S.A. 89:10537-10541; Wild, C. T. et al. (1994) “PEPTIDES CORRESPONDING TO A PREDICTIVE ALPHA-HELICAL DOMAIN OF HUMAN IMMUNODEFICIENCY VIRUS TYPE 1 GP41 ARE POTENT INHIBITORS OF VIRUS INFECTION,” Proc. Natl., Acad. Sci. U.S.A. 91:9770-9774: Chan, D. C. et al. (1998) “EVIDENCE THAT A PROMINENT CAVITY IN THE COILED COIL OF HIV TYPE 1 GP41 IS AN ATTRACTIVE DRUG TARGET,” Proc. Natl. Acad. Sci. U.S.A. 95:15613-15617; Eckert, D. M. et al. (2001) “DESIGN OF POTENT INHIBITORS OF HIV-1 ENTRY FROM THE GP41 N-PEPTIDE REGION,” Proc. Natl. Acad. Sci. U.S.A. 98:11187-11192). These peptides do not bind the fusion-active or postfusogenic state of gp41 as represented by the ectodomain of gp41 free in solution and are thought to interact with a pre-hairpin intermediate (Eckert, D. M. et al. (2001) “MECHANISMS OF VIRAL MEMBRANE FUSION AND ITS INHIBITION,” Annu. Rev. Biochem. 70:777-810; Furuta, R. A. et al. (1998) “CAPTURE OF AN EARLY FUSION-ACTIVE CONFORMATION OF HIV-1 GP41,” Nat. Struct. Biol. 5:276-279; Chan, D. C. et al. (1998) “HIV ENTRY AND ITS INHIBITION,” Cell 93:681-684) in which the N- and C-helices are not associated and the trimeric coiled-coil of N-helices is exposed (FIG. 1, Panel a, top left). Peptides derived from the C-terminal helix, such as C34 (residues 628-661 of HIV-1 Env) and T20 (residues 638-673 of HIV-1 Env; currently in phase III clinical trials (Kilby, J. M. et al. (1998) “POTENT SUPPRESSION OF HIV-1 REPLICATION HUMANS By T-20, A PEPTIDE INHIBITOR OF G41-MEDIATED VIRUS ENTRY,” Nat. Med. 4:1302-1307; Pozniak, A. (2001) “HIV FUSION INHIBITORS,” J. HIV Ther. 6:2-94) target the exposed face of the trimeric coiled-coil of N-helices (Chan, D. C. et al. (1998) “EVIDENCE THAT A PROMINENT CAVITY IN THE COILED COIL OF HIV TYPE 1 GP41 IS AN ATTRACTIVE DRUG TARGET,” Proc. Natl. Acad. Sci. U.S.A. 95:15613-15617; Furuta, R. A. et al. (1998) “CAPTURE OF AN EARLY FUSION-ACTIVE CONFORMATION OF HIV-1 GP41,” Nat. Struct. Biol. 5:276-279; Chan, D. C. et al. (1998) “HIV ENTRY AND ITS INHIBITION,” Cell 93:681-684; Weissenhorn, W. et al. (1999) “STRUCTURAL BASIS FOR MEMBRANE FUSION BY ENVELOPED VIRUSES,” Mol. Membr. Biol. 16:3-9; Kliger, Y. et al. (2000) “INHIBITION OF HIV-1 ENTRY BEFORE GP41 FOLDS INTO ITS FUSION-ACTIVE CONFORMATION,” J. Mol. Biol. 295:163-168). Engineered constructs such as the chimeric protein N_{CCG}-gp41 (Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489), which features an exposed, stable, disulfide-linked, trimeric coiled-coil of N-helices grafted onto the minimal, thermostable ectodomain of gp41; peptides in which the trimeric coiled-coil of N-helices is stabilized by fusion to the GCN4 trimeric coiled-coil (Eckert, D. M. et al. (2001) “DESIGN OF POTENT INHIBITORS OF HIV-1 ENTRY FROM THE GP41 N-PEPTIDE REGION,” Proc. Natl. Acad. Sci. U.S.A. 98:11187-11192); and the protein 5-helix (Root, M. J. et al. (2001) “PROTEIN DESIGN OF AN HIV-1 ENTRY INHIBITOR,” Science 291:884-888), in which the internal trimeric coiled-coil of N-helices is surrounded by only two C-helices, specifically target the C-region in the pre-hairpin intermediate state (FIG. 1, Panel a, top left). In both instances, packing of the C-region onto the trimeric coiled-coil of N-helices is blocked, and hairpin formation is inhibited. Although the ectodomain of gp41 in free solution is highly thermostable (with a Tₘ in excess of 100° C.) (Wingfield, P. T. et al. (1997) “THE EXTRACELLULAR DOMAIN OF IMMUNODEFICIENCY VIRUS GP41 PROTEIN: EXPRESSION IN ESCHERICHIA COLI, PURIFICATION, AND CRYSTALLIZATION,” Protein Sci. 6, 1653-1660), it has been shown to exist as a monomer-trimer equilibrium (Wingfield, P. T. et al. (1997) “THE EXTRACELLULAR DOMAIN OF IMMUNODEFICIENCY VIRUS GP41 PROTEIN: EXPRESSION IN ESCHERICIA COLI, PURIFICATION, AND CRYSTALLIZATION,” Protein Sci. 6, 1653-1660; Caffrey, M. et al. (1999) “MONOMER-TRIMER EQUILIBRIUM OF THE ECTODOMAIN OF SIV GP41: INSIGHT INTO THE MECHANISM OF PEPTIDE INHIBITION OF HIV INFECTION,” Protein Sci. 8:1904-1907). In the context of the fusion process, the trimeric coiled-coil of N-helices in the pre-hairpin intermediate state may also exist as a monomer-trimer equilibrium (Caffrey, M. et al. (1998) “THREE-DIMENSIONAL SOLUTION STRUCTURE OF THE 44 KDA ECTODOMAIN OF SIV GP41,” EMBO J. 17:4572-4584; Caffrey, M. et al. (1999) “MONOMER-TRIMER EQUILIBRIUM OF THE ECTODOMAIN OF SIV GP41: INSIGHT INTO THE MECHANISM OF PEPTIDE INHIBITION OF HIV INFECTION,” Protein Sci. 8:1904-1907; Peisajovich, S. G. et al; (2001) “SIV GP41 BINDS To MEMBRANES BOTH IN THE MONOMERIC AND TRIMERIC STATES: CONSEQUENCES FOR THE NEUROPATHOLOGY AND INHIBITION OF HIV INFECTION,” J. Mol. Biol. 311:249-254). If this is indeed the case, blocking the formation of the fusion-competent, homotrimeric coiled-coil of N-helices may provide another molecular target for inhibiting HIV cell fusion. In this article, we present the design and characterization of a peptide, derived from the N-helix of gp41, in which the sites of interaction with the C-helices have been mutated, but the sites of intermolecular contacts between the N-helices have been preserved. This peptide, which we term N36^{Mut(e,g)}, is about 50-fold more effective in inhibiting HIV Env-mediated cell fusion than the N36 peptide (residues 546-581 of HIV-1 Env) of gp41 from which it was derived. These data strongly suggest that the homotrimeric coiled-coil of N-helices in the pre-hairpin state can be disrupted.

The present invention additionally provides a method of treating immunodeficiency virus infection which comprises administering an effective amount of such inhibitor peptides of the present invention to a mammal either infected with such virus or at risk of such infection. Thus, the invention provides a treatment for: humans either infected with HIV or at risk of such infection; felines either infected with feline immunodeficiency virus or at risk of such infection; and simians either infected with simian immunodeficiency virus or at risk of such infection.

The effective amount of the inhibitor peptides of the present invention to be administered to the mammal, such as a human, is a subtoxic amount. Preferably, the subtoxic amount is an amount that produces little or no killing of uninfected cells. More preferably, the subtoxic amount is an amount that produces little or no effect on the morphology of uninfected cells. The inhibitor peptides of the present invention can be administered in a single dose or in multiple doses in a given period of time (e.g., a single daily dose or two or more doses a day). The subtoxic dose depends on the age, weight, general health, and extent of infection being treated. The inhibitor peptides of the present invention may be administered alone, or in combination with other immunodeficiency virus treatment regimens.

A composition is said to be “pharmacologically acceptable” if its administration can be tolerated by a recipient patient. An agent is physiologically significant if its presence results in a detectable change in the physiology of a recipient patient. The administration of such compounds may be for either a “prophylactic” or “therapeutic” purpose. The compositions of the present invention are said to be administered in a “therapeutically effective amount” if the amount administered is physiologically significant to provide a therapy for an actual infection. When provided therapeutically, the compound, is preferably provided at (or shortly after) the onset of a symptom of actual infection. The therapeutic administration of the compound serves to attenuate any actual infection. The compositions of the present invention are said to be administered in a “prophylactically effective amount” if the amount administered is physiologically significant to provide a therapy for an potential infection. When provided prophylactically, the compound is preferably provided in advance of any immunodeficiency virus infection or symptom thereof. The prophylactic administration of the compound serves to prevent or attenuate any subsequent infection.

The compounds of the present invention can be administered in conventional solid or liquid pharmaceutical administration forms, for example, as uncoated or (film-) coated tablets, capsules, powders, granules, suppositories or solutions. The active substances can, for this purpose, be processed with conventional pharmaceutical aids such as tablet binders, fillers, preservatives, tablet disintegrants, flow regulators, plasticizers, wetting agents, dispersants, emulsifiers, solvents, sustained release compositions, antioxidants and/or propellant gases. The therapeutic compositions obtained in this way typically contain from about 0.1% to about 90% by weight of the active substance.

In addition, the pharmaceutical composition can also contain pharmaceutically acceptable additives including, for example, diluents, binders, stabilizers, and preservatives. Administration of pharmaceutically acceptable salts described herein is preferred. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. Preferred salts include but are not limited to sodium phosphate, sodium acetate, sodium bicarbonate, sodium sulfate, sodium pyruvate, potassium phosphate, potassium acetate, potassium bicarbonate, potassium sulfate, potassium pyruvate, disodium DL-α-glycerol-phosphate, and disodium glucose-6-phosphate. “Phosphate” salts of sodium or potassium can be either the monobasic form, e.g., NaHPO₄, or the dibasic form, e.g., Na₂HPO₄, but a mixture of the two, resulting in a desired pH, is most preferred.

As used herein a “salt” is a substance produced from the reaction between acids and bases which comprises a metal (cation) and a nonmetal (anion). Salt crystals may be “hydrated” i.e., contain one or more water molecules. Such hydrated salts, when dissolved in an aqueous solution at a certain molar concentration, are equivalent to the corresponding anhydrous salt dissolved in an aqueous solution at the same molar concentration. For the present invention, salts which are readily soluble in an aqueous solution are preferred.

Further, the pharmaceutical composition may be prepared in the form of admixture with one or more pharmaceutically acceptable excipients so long as such additional excipients do not interfere with the effectiveness of the peptides and the side effects and adverse reactions are not increased additively or synergistically. The pharmaceutical compositions of the present invention can be associated with chemical moieties which may improve the composition's solubility, absorption, biological half life, etc. The moieties may alternatively decrease the toxicity of the pharmaceutical compositions, eliminate or attenuate any undesirable side effect of the pharmaceutical compositions, etc. Moieties capable of mediating such effects are disclosed in Remington's Pharmaceutical Sciences, 19^{th} Edition, A. R. Gennaro, ed., Mack Publishing Co., Easton, Pa. (1995). Procedures for coupling such moieties to a molecule are well known in the art.

As used herein a pharmaceutical “excipient” is a substance other than the pharmacologically active drug or prodrug which is included in the manufacturing process or are contained in a finished pharmaceutical product dosage form. Some, for example, comprise the product's delivery system. In the preferred embodiment pharmaceutical excipients transport the active drug to the site in the body where the drug is intended to exert its action. In more preferred embodiment, excipients will keep the drug from being released too early in the assimilation process in places where it could damage tender tissue and create gastric irritation or stomach upset. In even more preffered embodiment, excipients will help the drug to disintegrate into particles small enough to reach the blood stream more quickly and still others protect the product's stability so it will be at maximum effectiveness at time of use. In order to improve patient compliance, these excipients can be used simply to make the pharmaceutical composition taste and look better (International Pharmaceutical Excipients Council of the Americas

http://www.ipecamericas.org/public/faqs).

Suitable excipients include Magnesium Stearate, Lactose, Microcrystalline Cellulose, Starch (corn), Silicon Dioxide, Titanium Dioxide, Stearic Acid, Sodium Starch Glycolate, Gelatin, Talc, Sucrose, Calcium Stearate, Povidone, Pregelatinized Starch, Hydroxy Propyl Methylcellulose, OPA products (coatings & inks), Croscarmellose, Hydroxy Propyl Cellulose, Ethylcellulose, Calcium Phosphate (dibasic), Crospovidone, Shellac (and Glaze).

The pharmaceutical compositions of the present invention may be administered by any suitable means, for example, inhalation, or interdermally, intracavity (e.g., oral, vaginal, rectal, nasal, peritoneal, ventricular, or intestinal), intradermally, intramuscularly, intranasally, intraocularly, intraperitoneally, intrarectally, intratracheally, intravenously, orally, subcutaneously, transdermally, or transmucosally (i.e., across a mucous membrane) in a dose effective for the production of neutralizing antibody and resulting in protection from infection or disease. The present pharmaceutical compositions can generally be administered in the form of a spray for intranasal administration, or by nose drops, inhalants, swabs on tonsils, or a capsule, liquid, suspension or elixirs for oral administration. The pharmaceutical compositions may be in the form of single dose preparations or in multi-dose flasks. Reference is made to Remington's Pharmaceutical Sciences, 19^{th} Edition, A. R. Gennaro, ed., Mack Publishing Co., Easton, Pa. (1995).

Administration can be into one or more tissues including but not limited to muscle, skin, brain, lung, liver, spleen, bone marrow, thymus, heart, e.g., myocardium, endocardium, and pericardium, lymph nodes, blood, bone, cartilage, pancreas, kidney, gall bladder, stomach, intestine, testis, ovary, uterus, rectum, nervous system, eye, gland, or connective tissue. Furthermore, in the methods of the present invention, the pharmaceutical compositions may be administered to any internal cavity of a mammal, including, but not limited to, the lungs, the mouth, the nasal cavity, the stomach, the peritoneal cavity, the intestine, any heart chamber, veins, arteries, capillaries, lymphatic cavities, the uterine cavity, the vaginal cavity, the rectal cavity, joint cavities, ventricles in brain, spinal canal in spinal cord, and the ocular cavities. Administration may be by needle injection, catheter infusion, biolistic injectors, particle accelerators (e.g., pneumatic “needleless” injectors), gelfoam sponge depots, other commercially available depot materials (e.g., hydrogels), osmotic pumps (e.g., Alza minipumps), oral or suppositorial solid (tablet or pill) pharmaceutical formulations, topical skin creams, and decanting, use of polynucleotide coated suture (Qin, J. Y. et al. (1999) “GENE SUTURE—A NOVEL METHOD FOR INTRAMUSCULAR GENE TRANSFER AND ITS APPLICATION IN HYPERTENSION THERAPY,” Life Sciences 65:2193-2203) or topical applications during surgery. Any mode of administration can be used so long as the mode results prophylactic or therapeutic efficacy. Methods to detect such a response include serological methods, e.g., western blotting, staining tissue sections by immunohistochemical methods, and measuring the activity of the polypeptide.

In one embodiment, DNA compositions will be used to provide the preferred peptides of the present invention. Pharmaceutical DNA compositions and methods for their manufacture and delivery that may be used in accordance with the present invention are disclosed in U.S. Pat. Nos. 5,589,466; 5,620,896; 5,641,665; 5,703,055; 5,707,812; 5,846,946; 5,861,397; 5,891,718; 6,022,874; 6,147,055; 6,214,804; 6,228,844; 6,399,588; 6,413,942; 6,451,769, European, Patent Documents EP1165140A2; EP1006796A1 and EP0929536A1; and PCT Patent Publications WO00/57917, WO00/73263; WO01/09303; WO03/028632; WO94/29469; WO95/29703; and WO98/14439.

The compositions of the present invention can be lyophilized to produce pharmaceutical compositions in a dried form for ease in transportation and storage. The pharmaceutical compositions of the present invention may be stored in a sealed vial, ampoule or the like. In the case where the pharmaceutical composition is in a dried form, the composition is dissolved or suspended (e.g., in sterilized distilled water) before administration. An inert carrier such as saline or phosphate buffered saline or any such carrier in which the pharmaceutical compositions has suitable solubility, may be used.

The pharmaceutical compositions can be solubilized in a buffer prior to administration. Suitable buffers include, for example, phosphate buffered saline (PBS), normal saline, Tris buffer, and sodium phosphate vehicle (100-150 mM preferred). Insoluble polynucleotides can be solubilized in a weak acid or base, and then diluted to the desired volume with a neutral buffer such as PBS. The pH of the buffer is suitably adjusted, and moreover, a pharmaceutically acceptable additive can be used in the buffer to provide an appropriate osmolarity within the lipid vesicle. Preferred salt solutions and auxiliary agents are disclosed herein.

Compositions used in of the present invention can be formulated according to known methods. Suitable preparation methods are described, for example, in Remington's Pharmaceutical Sciences, 19^{th} Edition, A. R. Gennaro, ed., Mack Publishing Co., Easton, Pa. (1995), incorporated herein by reference in its entirety. Although the composition is preferably administered as an aqueous solution, it can be formulated as an emulsion, gel, solution, suspension, lyophilized form, or any other form known in the art. According to the present invention, if the composition is formulated other than as an aqueous solution, it will require resuspension in an aqueous solution prior to administration.

Such compositions may be formulated into any of the various compositions and may be used in any of the methods disclosed herein. For aqueous compositions used in vivo, use of sterile pyrogen-free water is preferred. Such formulations will contain an effective amount of such peptide together with a suitable salt and/or pharmaceutically acceptable excipient as disclosed herein, in order to prepare pharmaceutically acceptable compositions suitable for optimal administration to a vertebrate.

The effective amount of a peptide, or a pharmaceutically acceptable salt thereof included in a pharmaceutical composition depends on factors including the age and weight of the subject, the delivery method and route, the type of treatment desired, and the type of peptide or composition being administered. In general, a pharmaceutical composition of the present invention that includes peptides or peptide compositions will contain from about 1 ng to about 30 mg of such peptides or peptide compositions, more preferably, from about 100 ng to about 10 mg of such peptides or peptide compositions. Certain preferred compositions of the present invention may include about 1 ng of such peptides or peptide compositions, about 5 ng of such peptides or peptide compositions, about 10 ng of such peptides or peptide compositions, about 50 ng of such peptides or peptide compositions, about 100 ng of such peptides or peptide compositions, about 500 ng of such peptides or peptide compositions, about 1 μg of such peptides or peptide compositions, about 5 μg of such peptides or peptide compositions, about 10 μg of such peptides or peptide compositions, about 50 μg of such peptides or peptide compositions, about 100 μg of such peptides or peptide compositions, about 150 μg of such peptides or peptide compositions, about 200 μg of such peptides or peptide compositions, about 250 μg of such peptides or peptide compositions, about 300 μg of such peptides or peptide compositions, about 350 μg of such peptides or peptide compositions, about 400 μg of such peptides or peptide compositions, about 450 μg of such peptides or peptide compositions, about 500 μg of such peptides or peptide compositions, about 550 μg of such peptides or peptide compositions, about 600 μg of such peptides or peptide compositions, about 650 μg of such peptides or peptide compositions, about 700 μg of such peptides or peptide compositions, about 750 μg of such peptides or peptide compositions, about 800 μg of such peptides or peptide compositions, about 850 μg of such peptides or peptide compositions, about 900 μg of such peptides or peptide compositions, about 950 μg of such peptides or peptide compositions, about 1 mg of such peptides or peptide compositions, about 5 mg of such peptides or peptide compositions, about 10 mg of such peptides or peptide compositions, about 15 mg of such peptides or peptide compositions, about 20 mg of peptides or peptide compositions, about 25 mg of such peptides or peptide compositions, or about 30 mg of such peptides or peptide compositions.

In one embodiment of the present invention, alternatively, or conjunctively, one or more of the above-described pharmaceutical compositions will comprise a peptide or peptide composition in admixture with one or more pharmaceutically acceptable excipients that may be administered to a recipient prior to the commencement of HIV infection, or subsequent to the onset of such infection. In accordance with the methods of the present invention, a single antiretroviral pharmaceutical composition, peptide or peptide composition containing more than one peptide sequence may be administered. Alternatively, more than one peptide, peptide composition, pharmaceutically acceptable salt thereof or pharmaceutically acceptable composition may be co-administered or sequentially administered.

In particular, the peptides of the present invention may be provided to recipients alone or in combination with one or more other pharmaceutical compositions (such as those that inhibit the binding of HIV to cell membranes, those that inhibit HIV reverse transcriptase, those that inhibit the exit of the virus from the host cell and/or the infection of new cellular targets, or those that inhibit viral enzymes (e.g., drugs such as 2′,3′-dideoxynucleoside analogs (e.g., azidothymidine (AZT), dideoxyinosine, dideoxycytidine, and d4thymidine, etc.)). In preferred embodiments, the peptides of the present invention may be provided to recipients in combination with one or more other pharmaceutical compositions that inhibit retroviral protease (especially HIV protease), retroviral reverse transcriptase (especially HIV reverse transcriptase), retroviral fusion (especially HIV fusion) and/or retroviral integrase (especially HIV integrase) (Clumeck, N. et al. (1993) “CURRENT USE OF ANTI-HIV DRUGS IN AIDS,” J. Antimicrob. Chemother. (Suppl. A) 32, 133-138; Witvrouw, M. et al. (2003) “THE INTEGRASE OF THE HUMAN IMMUNODEFICIENCY VIRUS AS A NOVEL TARGET FOR THE ANTIVIRAL THERAPY OF AIDS,” Verh K Acad Geneeskd Belg 65(5): 325-34; Docherty, A. J. et al. (2003) “PROTEASES AS DRUG TARGETS,” Biochem Soc Symp. (70): 147-61; Nair, V. (2003) “NOVEL INHIBITORS OF HIV INTEGRASE: THE DISCOVERY OF POTENTIAL ANTI-HIV THERAPEUTIC AGENTS,” Curr Pharm Des.: 9(31): 2553-65; Thaker, H. K. et al. (2003) “HIV VIRAL SUPPRESSION IN THE ERA OF ANTIRETROVIRAL THERAPY,” Postgrad Med J. 79(927): 36-42; De Clercq, E. (2002) “NEW ANTI-HIV AGENTS AND TARGETS,” Med. Res. Rev. 6:531-65; Mitsuya, H. et al. (1987) “STRATEGIES FOR ANTIVIRAL THERAPY IN AIDS,” Nature 325(6107): 773-8; Gallo, R. C. et al. (1987) “ETIOLOGY OF AIDS RESEARCH,” Nature 326(6112): 435-6; “PROTEASES INHIBITORS AND BEYOND,” PI Perspective 21:16-16 (1997) (Anonymous); James, J. S. (2002) “RETROVIRUSES CONFERENCE: SOME NEW DRUGS IN THE PIPELINE,” AIDS Treat News 12 (379):24). Examples of protease inhibitors include: Amperanir (APV) (Agenerase; GlaxoSmithKline/Vertex); Atazanavir Sulfate (Reyataz; Bristol-Myers Squibb); Fosamprenavir Calcium(Lexiva; GlaxoSmithKline); Indinavir (IDV) (Crixivan; Merck); Lopinavir/ritonavir (LPV/r) (Kaletra; Abbott Laboratories); Nelfinavir (NFV) (Viracept; Agouron Pharmaceuticals); Ritonavir (RTV) (Norvir; Abbott Laboratories); Saquinavar mesylate (Invirase; Hoffmann-La Roche); and Saquinavir (SQV) (Fortovase; Hoffmann-La Roche). Examples of nucleoside reverse transcriptase inhibitors include: Abacavir (Ziagen; GlaxoSmithKline); Abacavir, Zidovudine, And Lamivudine (Trizivir, GlaxoSmithKline); Didanosine, ddl, Dideoxyinosine (Videx; Bristol Myers-Squibb); Enteric Coated Didanosine (Videx Ec; Bristol Myers-Squibb); FTC, Emtricitabine (Emtriva; Gilead Sciences); Lamivudine And Zidovudine (Combivir; GlaxoSmithKline); Lamivudine, 3TC (Epivir; GlaxoSmithKline); Nelfinavir Mesylate, NFV (Viracept; Agouron Pharmaceuticals); Stavudine, D4t (Zerit; Bristol Myers-Squibb); Tenofovir Disoproxil Fumarate (Viread; Gilead); Zalcitabine, Ddc, Dideoxycytidine (Hivid; Hoffmann-La Roche); and Zidovudine, AZT, Azidothymidine, ZDV (Retrovir GlaxoSmithKline). Examples of non-nucleoside reverse transcriptase inhibitors include: Delaviridine (DLV) (Rescriptor; Pharmacia); Efavirenz (EVF) (Sustiva; Bristol-Myers Squibb); and Nevirapine (NVP) (Viramune; Boehringer Ingelheim). Examples of fusion inhibitors include: Enfuvirtide; T-20 (Fuzeon; Hoffmann-La Roche/Trimeris) (O'Brien, W. A. (2003) “NEW CLASSES OF HIV DRUGS ON THE HORIZON,” AIDS Read. 13(3 Suppl.):S4-8;

http://www.fda.gov/oashi/aids/pedlbl.html)

The present invention also provides kits for use in treating retroviral infection comprising an administration means and a container means containing a pharmaceutical composition of the present invention. Preferably, the container in which the composition is packaged prior to use will comprise a hermetically sealed container enclosing an amount of the lyophilized formulation or a solution containing the formulation suitable for a pharmaceutically effective dose thereof, or multiples of an effective dose. The composition is packaged in a sterile container, and the hermetically sealed container is designed to preserve sterility of the pharmaceutical formulation until use. Optionally, the container can be associated with administration means and/or instruction for use.

Having now generally described the invention, the same will be more readily understood through reference to the following examples, which are provided by way of illustration, and are not intended to be limiting of the present invention, unless specified.

### EXAMPLE 1

### Experimental Methods

Peptides—All peptides (FIG. 2, Panel b), purchased from Commonwealth Biotechnologies (Richmond, Va.), were synthesized on a solid phase support, purified by reverse phase high pressure liquid chromatography, and verified for purity by mass spectrometry and amino acid composition. All peptides bear an acetyl group at the N terminus and an amide group at the C terminus. Concentrations of peptides were determined spectrophotometrically: the calculated A₂₈₀ values (1-cm path length) for a concentration of 1 mg/ml N36, N36^{Mut(e,g)}, N36^{Mut(a,d)}, and C34 are 1.35, 1.31, 1.34, and 2.90, respectively. The corresponding molecular masses are 4160, 4293, 4182, and 4286 Da, respectively.

As used herein, the “N36^{Mut(a,d)}” has the amino
acid sequence:

(SEQ ID NO:4)

SGIVQQLNNQ LRAEEANQHL EQLSVWGSKQ NQARRL
1 10 20 30 36

Circular Dichroism—CD spectra of peptides (at a concentration corresponding to 0.7-0.8 A₂₈₀) were recorded at 25° C. on a JASCO J-720 spectropolarimeter using a 0.05-cm path length cell. Quantitative evaluation of secondary structure from the CD spectrum was carried out using the program CDNN (www.bioinformatik.biochemtech.uni-halle.de/cd_spect/index.html; Bohm, G. et al. (1992) “QUANTITATIVE ANALYSIS OF PROTEIN FAR UV CIRCULAR DICHROISM SPECTRA BY NEURAL NETWORKS,” Protein Eng. 5:191-195).

Sedimentation Equilibrium—Sedimentation equilibrium experiments were conducted at 20.0° C. and three different rotor speeds (16,000, 20,000, and 24,000) on a Beckman Optima XL-A analytical ultracentrifuge. Peptide samples were prepared in 50 mM sodium formate buffer (pH=3) and loaded into the ultracentrifuge cells at nominal loading concentrations of ˜0.2 and 0.7-0.8 A₂₈₀. Data were analyzed in terms of a single ideal solute to obtain the buoyant molecular mass, M(1−νρ), using the Optima XL-A data analysis software (Beckman). The value for the experimental molecular mass M was determined using calculated values for the density ρ (determined at 20° C. using standard tables) and partial specific volume ν (calculated on the basis of amino acid composition (Perkins, S. J. (19586) “PROTEIN VOLUMES AND HYDRATION EFFECTS. THE CALCULATIONS OF PARTIAL SPECIFIC VOLUMES, NEUTRON SCATTERING MATCHPOINTS AND 280-NM ABSORPTION COEFFICIENTS FOR PROTEINS AND GLYCOPROTEINS FROM AMINO ACID SEQUENCES,” Eur. J. Biochem. 157:169-180)).

Cell Fusion Assay—Inhibition of HIV Env-mediated cell fusion by peptides was carried out as described previously (Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489) using a modification (Salzwedel, K. et al. (2000) “SEQUENTIAL CD4-CORECEPTOR INTERACTIONS IN HUMAN IMMUNODEFICIENCY VIRUS TYPE 1 ENV FUNCTION: SOLUBLE CD4 ACTIVATES ENV FOR CORECEPTOR-DEPENDENT FUSION AND REVEALS BLOCKING ACTIVITIES OF ANTIBODIES AGAINST CRYPTIC CONSERVED EPITOPES ON GP120,” J. Virol. 74:326-333) of the vaccinia virus-based reporter gene assay (using soluble CD4 at a final concentration of 200 nM). B-SC-1 cells were used for both target and effector cell populations. Target cells were co-infected with vCB21R-LacZ and vCBYF1-fusin (CXCR4), and effector cells were co-infected with vCB41 (Env) and vP11T7gene1 at a multiplicity of infection of 10. For inhibition studies, peptides were added to an appropriate volume of Dulbecco's modified Eagle's medium (2.5%) and phosphate-buffered saline to yield identical buffer compositions (100 μl) followed by addition of 1×10⁵ effector cells (in 50 μl of medium) per well. After incubation for 15 min, 1×10⁵ target cells (in 50 μl) and soluble CD4 were added to each well. Following 2.5 h of incubation, β-galactosidase activity of cel> lysates was measured (A₅₇₀; Molecular Devices 96-well spectrophotometer) upon addition of chlorophenol red-βD-galactopyranoside (Roche Molecular Biochemicals). The curves for % fusion versus peptide inhibitor concentration were fit by nonlinear least-squares optimization using the program FACSIMILE (Chance, E. M. et al. (1979) FACSIMILE, Atomic Energy Research Establishment Report R8775, Harwell, H. M. Stationary Office, London, UK; Clore, G. M. (1983) In: COMPUTING IN BIOLOGICAL SCIENCE (Geisow, M. J., and Barrett, A. N., eds), pp. 313-348, Elsevier/North-Holland, N.Y.).

### EXAMPLE 2

### Design of Peptide Inhibitors

The helical wheel diagram in FIG. 2, Panel a illustrates the interactions between the N-helices and between the N- and C-helices as observed in both the NMR (Caffrey, M. et al. (1998) “THREE-DIMENSIONAL SOLUTION STRUCTURE OF THE 44 KDA ECTODOMAIN OF SIV GP41,” EMBO J. 17:4572-4584) and x-ray (Chan, D. C. et al. (1997) “CORE STRUCTURE OF GP41 FROM THE HIV ENVELOPE GLYCOPROTEIN,” Cell 89:263-273; Weissenhorn, W. et al. (1997) “ATOMIC STRUCTURE OF THE ECTODOMAIN FROM HIV-1 GP41,” Nature 387:426-430; Tan, K. J. et al. (1997) “ATOMIC STRUCTURE OF A THERMOSTABLE SUBDOMAIN OF HIV-1 GP41,” Proc. Natl. Acad. Sci. (U.S.A.) 94:12303-12308; Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139) structures of the fusogenic/postfusogenic state of the ectodomain of gp41. Internal contacts between the N-helices involve positions a and d of the helical wheel (Chan, D. C. et al. (1997) “CORE STRUCTURE OF GP41 FROM THE HIV ENVELOPE GLYCOPROTEIN,” Cell 89:263-273). Each C-helix interacts with two N-helices (one intra- and the other intersubunit): these contacts involve positions e and g of the N-helices and positions a and d of the C-helix (Chan, D. C. et al. (1997) “CORE STRUCTURE OF GP41 FROM THE HIV ENVELOPE GLYCOPROTEIN,” Cell 89:263-273).

The first crystal structure of the HIV-1 gp41 ectodomain core consisted of a complex of N36 and C34 peptides comprising residues 546-581 and 628-661, respectively, of HIV-1 Env (Chan, D. C. et al. (1997) “CORE STRUCTURE OF GP41 FROM THE HIV ENVELOPE GLYCOPROTEIN,” Cell 89:263-273). Using the N36 and C34 peptides as starting points, we designed two peptides: N36^{Mut(e,g)}, which can only undergo self-association but cannot interact with C34, and N36^{Mut(a,d)}, which can no longer self-associate but could potentially still interact with C34 (FIG. 2, Panel b). In the case of N36^{Mut(e,g)}, the residues at positions e and g of N36 have been replaced by residues at positions e and g of C34. Since the latter residues are located on the external surface of C34 in the context of the ectodomain gp41 core (Caffrey, M. et al. (1998) “THREE-DIMENSIONAL SOLUTION STRUCTURE OF THE 44 KDA ECTODOMAIN OF SIV GP41,” EMBO J. 17:4572-4584; Chan, D. C. et al. (1997) “CORE STRUCTURE OF GP41 FROM THE HIV ENVELOPE GLYCOPROTEIN,” Cell 89:263-273; Weissenhorn, W. et al. (1997) “ATOMIC STRUCTURE OF THE ECTODOMAIN FROM HIV-1 GP41,” Nature 387:426-430; Tan, K. J. et al. (1997) “ATOMIC STRUCTURE OF A THERMOSTABLE SUBDOMAIN OF HIV-1 GP41,” Proc. Natl. Acad. Sci. (U.S.A.) 94:12303-12308; Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139) and since C34 on its own is monomeric (Lu, M. et al. (1995) “A TRIMERIC STRUCTURAL DOMAIN OF THE HIV-1 TRANSMEMBRANE GLYCOPROTEIN,” Nat. Struct. Biol. 2:1075-1082), this set of substitutions will prevent any interaction between N36^{Mut(e,g)} and the C-region of gp41 in its pre-hairpin intermediate state while preserving the intermolecular contacts required to form the trimeric coiled-coil of N-helices. In the case of N36^{Mut(a,d)}, the residues at positions a and d of N36 have been substituted by residues at positions f and c of C34, which are located on the solvent-exposed face of the ectodomain core of gp41 (Caffrey, M. et al. (1998) “THREE-DIMENSIONAL SOLUTION STRUCTURE OF THE 44 KDA ECTODOMAIN OF SIV GP41,” EMBO J. 17:4572-4584; Chan, D. C. et al. (1997) “CORE STRUCTURE OF GP41 FROM THE HIV ENVELOPE GLYCOPROTEIN,” Cell 89:263-273; Weissenhorn, W. et al. (1997) “ATOMIC STRUCTURE OF THE ECTODOMAIN FROM HIV-1 GP41,” Nature 387:426-430; Tan, K. J. et al. (1997) “ATOMIC STRUCTURE OF A THERMOSTABLE SUBDOMAIN OF HIV-1 GP41,” Proc. Natl. Acad. Sci. (U.S.A.) 94:12303-12308; Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139), thereby removing the intermolecular contacts required to form the trimeric coiled-coil of N-helices.

### EXAMPLE 3

### Biophysical Characterization of N36^{Mut(e,g)} and N36^{Mut(a,d)}

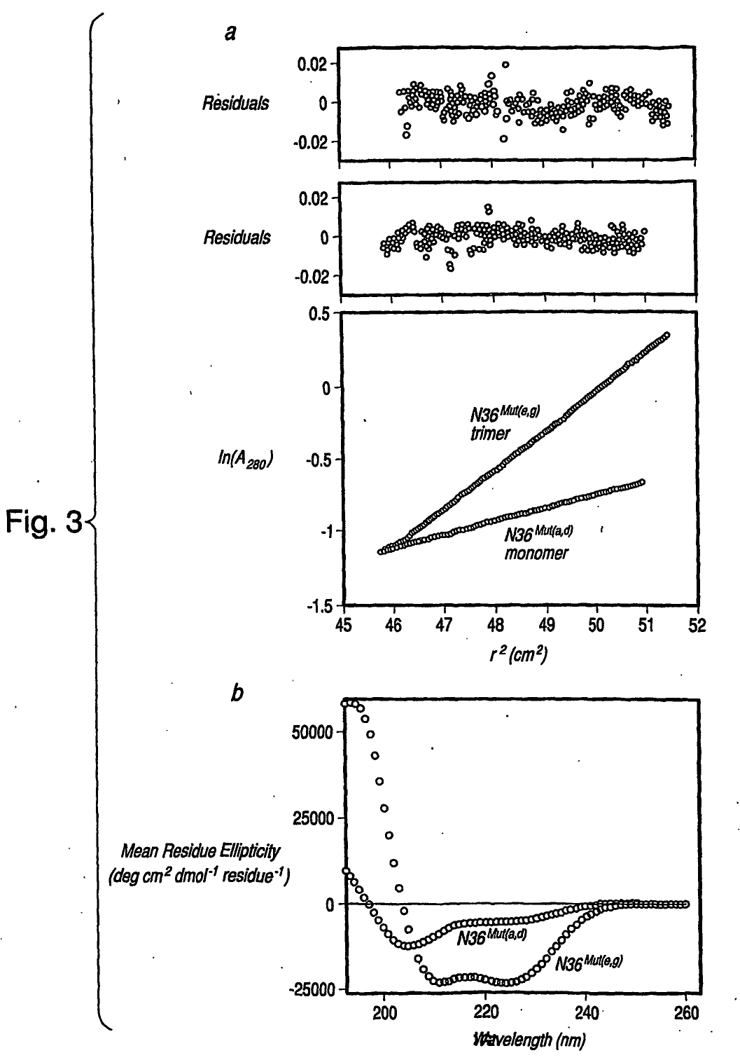

The results of analytical ultracentrifugation on N36^{Mut(e,g)} and N36^{Mut(a,d)} are presented in FIG. 3, Panel a. N36^{Mut(e,g)} behaves as a single monodisperse species at concentrations of ˜36 μM (in monomer; A₂₈₀˜0.2) and ˜124 μM (in monomer; A₂₈₀˜0.7) with a molecular mass of ˜12,000-12,500 Da, corresponding to a trimer. In this context it is worth noting that N36 on its own aggregates and does not form a well defined trimer (Eckert, D. M. et al. (2001) “DESIGN OF POTENT INHIBITORS OF HIV-1 ENTRY FROM THE GP41 N-PEPTIDE REGION,” Proc. Natl. Acad. Sci. U.S.A. 98:11187-11192), presumably due to further self-association involving the predominantly hydrophobic residues at positions e and g, which have been substituted by predominantly hydrophilic residues in N36^{Mut(e,g)} (FIG. 2, Panel b). N36^{Mut(a,d)} also behaves as a single monodisperse species at a concentration of ˜140 μM (A₂₈₀˜0.8), but its molecular mass is only ˜3700 Da, corresponding to a monomer.

CD spectra of N36^{Mut(e,g)} and N36^{Mut(a,d)} are shown in FIG. 3, Panel b, N36^{Mut(e,g)} displays a double minimum at 208 and 222 nm, characteristic of an α-helix> and quantification of the CD data (Bohm, G. et al. (1992) “QUANTITATIVE ANALYSIS OF PROTEIN FAR UV CIRCULAR DICHROISM SPECTRA BY NEURAL NETWORKS,” Protein Eng. 5:191-195) indicates a helical content of ˜80%. N36^{Mut(a,d)}, on the other hand, is largely random coil (characterized by a minimum around 200 nm) with a small amount of α-helix (˜20%).

No evidence of interaction between either N36^{Mut(e,g)} or N36^{Mut(a,d)} and C34 was detected by either analytical ultracentrifugation or CD. The absence of interaction between N36^{Mut(e,g)} and C34 is exactly as predicted from the design since the points of contact with C34 have been mutated (cf. FIG. 2). The absence of interaction between N36^{Mut(a,d)} and C34 was initially somewhat surprising since the residues that contact C34 in the context of the fusogenic/postfusogenic state of the gp41 ectodomain were preserved. This result therefore indicates that C34 can only form a complex with a stable trimeric coiled-coil of N-helices. From a structural standpoint, this is readily understood since each C-helix contacts two N-helices of the trimeric coiled-coil (one intramolecular and the other intersubunit; cf. FIG. 2, panel a), and the buried surface area for each of the two interactions is comparable.

To exclude the remote possibility that N36^{Mut(e,g)} could behave in a manner analogous to C34 and bind to the surface of the trimeric coiled-coil of N-helices in the pre-hairpin intermediate of gp41, the interaction of N36^{Mut(e,g)} with the engineered protein N_{CCG}-gp41 was examined. N_{CCG}-gp41 is a chimeric protein that features an exposed trimeric coiled-coil of N-helices that is stabilized both by fusion to a minimal thermostable ectodomain of gp41 and by engineered intersubunit disulfide bonds (Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489). The exposed trimeric coiled-coil of N-helices in N_{CCG}-gp41 mimics that of the pre-hairpin intermediate of gp41, but in contrast to native gp41, the N-helices cannot dissociate since they are covalently tethered by disulfide bonds. Analytical ultracentrifugation on various mixtures of N36^{Mut(e,g)} and N_{CCG}-gp41 in ratios of 4.5:1 and 11.7:1 (24 μM N36^{Mut(e,g)} plus 5.3 μM N_{CCG}-gp41 and 51 μM N36^{Mut(e,g)} plus 4.4 μM N_{CCG}-gp41, respectively, with concentrations expressed in trimer) provided no evidence of any interactions between these two molecules, and the data were readily accounted for by a mixture of two ideal species.

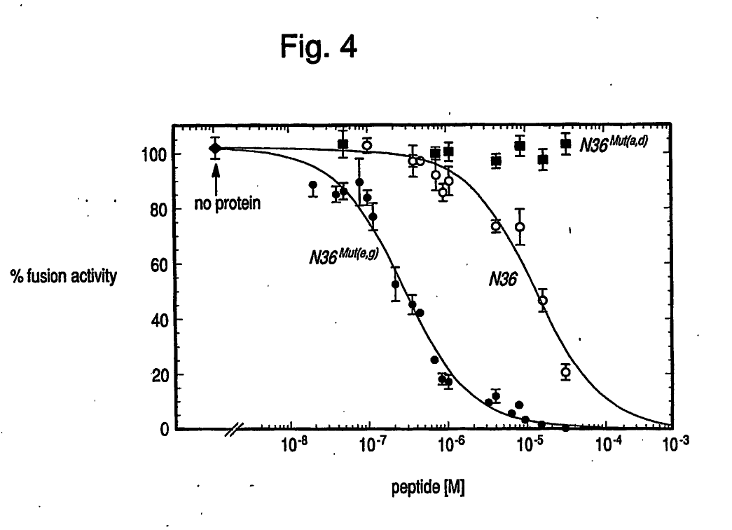

Inhibition of HIV Env-mediated Cell Fusion—The results of a quantitative vaccinia virus-based reporter gene assay (Salzwedel, K. et al. (2000) “SEQUENTIAL CD4-CORECEPTOR INTERACTIONS IN HUMAN IMMUNODEFICIENCY VIRUS TYPE 1 ENV FUNCTION: SOLUBLE CD4 ACTIVATES ENV FOR CORECEPTOR-DEPENDENT FUSION AND REVEALS BLOCKING ACTIVITIES OF ANTIBODIES AGAINST CRYPTIC CONSERVED EPITOPES ON GP120,” J. Virol. 74:326-333) for HIV Env-mediated cell fusion are shown in FIG. 4. N36 inhibits fusion with an IC₅₀ of 16±2 μM in agreement with previous results (Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489). N36^{Mut(e,g)} inhibits fusion with an IC₅₀ 308±22 nM. Thus N36^{Mut(e,g)} is ˜50-fold more active in inhibiting fusion than N36. N36^{Mut(a,d)}, on the other hand, fails to inhibit fusion even at concentrations as high as 0.1 mM. The lack of any fusion-inhibitory activity for N36^{Mut(a,d)} is exactly as predicted from the biophysical data since N36^{Mut(a,d)} does not self-associate and does not interact with C34.

Since N36^{Mut(e,g)} forms a well defined trimeric species that does not interact with either C34 or the chimeric protein N_{CCG}-gp41 (in which the N-helices of the solvent-exposed trimeric coil-coil are covalently linked by interhelical disulfide bonds), it must target the N-region of the pre-hairpin intermediate by forming fusion-incompetent heterotrimers (FIG. 1, panel b). Analytical ultracentrifugation on the ectodomain of gp41 indicates the presence of only monomer and trimer species in equilibrium (Wingfield, P. T. et al. (1997) “THE EXTRACELLULAR DOMAIN OF IMMUNODEFICIENCY VIRUS GP41 PROTEIN: EXPRESSION IN ESCHERICHIA COLI, PURIFICATION, AND CRYSTALLIZATION,” Protein Sci. 6, 1653-1660; Caffrey, M. et al. (1999) “MONOMER-TRIMER EQUILIBRIUM OF THE ECTODOMAIN OF SIV GP41: INSIGHT INTO THE MECHANISM OF PEPTIDE INHIBITION OF HIV INFECTION,” Protein Sci. 8:1904-1907); that is, assembly of the trimer is a highly cooperative process. The fusion-inhibitory activity of N36^{Mut(e,g)} therefore indicates the presence of a dynamic equilibrium between monomeric and trimeric forms of membrane-bound gp41 that allows subunit exchange to take place in the pre-hairpin intermediate state. The rate of exchange between these species must be sufficiently fast to permit efficient heterotrimer formation within the lifetime (˜15 min) of the pre-hairpin intermediate (Furuta, R. A. et al. (1998) “CAPTURE OF AN EARLY FUSION-ACTIVE CONFORMATION OF HIV-1 GP41,” Nat. Struct. Biol. 5:276-279, Jones, P. L. et al. (1998) “CONFORMATIONAL CHANGES IN CELL SURFACE HIV-1 ENVELOPE GLYCOPROTEINS ARE TRIGGERED BY COOPERATION BETWEEN CELL SURFACE CD4 AND CO-RECEPTORS,” J. Biol. Chem. 273:404-409; Muñoz-Barroso, I. et al. (1998) “DILATION OF THE HUMAN IMMUNODEFICIENCY VIRUS-1 ENVELOPE GLYCOPROTEIN FUSION PORE REVEALED BY THE INHIBITORY ACTION OF A SYNTHETIC PEPTIDE FROM GP41,” J. Cell Biol. 140, 315-323).

### EXAMPLE 4

### Modeling Inhibition of HIV Env-mediated Cell Fusion by N36^{Mut(e,g)}

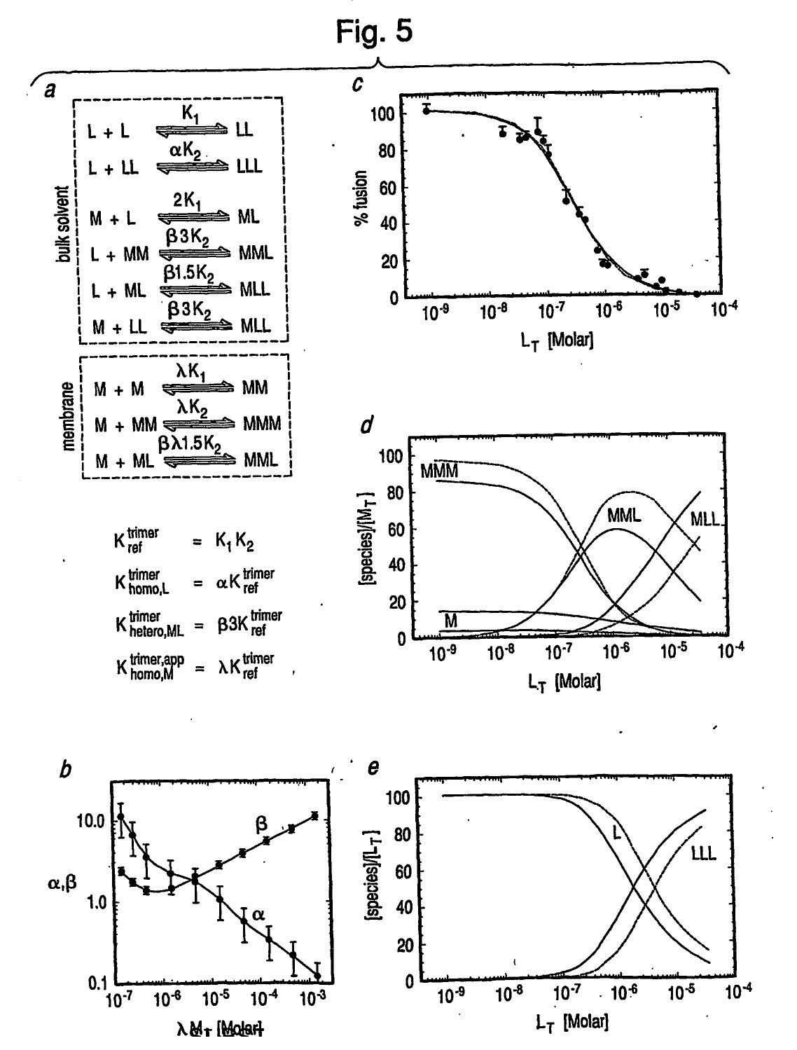

The inhibition curve for N36^{Mut(e,g)} is well fit by a simple Langmuir isotherm given by % fusion 100/(1+[N36^{Mut(e,g)}]/IC₅₀) (FIG. 4). Yet, mechanistically, the interaction of N36^{Mut(e,g)} with the pre-hairpin intermediate of gp41 is far more complex, involving multiple species in different homo- and hetero-oligomerization states. The simplest scheme describing the situation is presented in FIG. 5, Panel a. L and M represent N36^{Mut(e,g)} and the pre-hairpin intermediate of gp41 in their monomeric forms, respectively; LL and MM are homodimers; ML is a heterodimer; LLL and MMM are homotrimers; and MML and MLL are heterodimers. It is assumed that only the homotrimer MMM is fusion-active, and the fraction fusion activity is given by the ratio of [MMM]_{LT}/[MMM]_{LT=0}. The interactions between ligand (in its various oligomerization states) and membrane-bound protein (in its various homo- and hetero-oligomeric states) are described by their respective bulk solution concentrations. The interactions involving only membrane-bound species, however, are dependent on their concentrations in the two-dimensional membrane (i.e. number of molecules per unit area) that are much higher than their concentrations in bulk solvent. In terms of thermodynamics, all equilibria in FIG. 5, Panel a can be related to the species concentrations in bulk solvent by multiplying the relevant equilibrium constants by a factor λ to yield appropriate apparent equilibrium constants (FIG. 5, Panel a, middle).

The measured equilibrium association constant K_{ref}^{trimer} for the ectodomain of HIV-1 gp41 in free solution (i.e. the trimer of hairpins) is 4.8×10¹¹ M⁻² and is given by the product of the equilibrium association constants K₁ (monomer-dimer equilibrium) and K₂ (dimer-trimer equilibrium) (FIG. 5, Panel a, bottom). Since trimerization of the gp41 ectodomain is highly cooperative (Wingfield, P. T. et al. (1997) “THE EXTRACELLULAR DOMAIN OF IMMUNODEFICIENCY VIRUS GP41 PROTEIN: EXPRESSION IN ESCHERICHIA COLI, PURIFICATION, AND CRYSTALLIZATION,” Protein Sci. 6, 1653-1660; Caffrey, M. et al. (1999) “MONOMER-TRIMER EQUILIBRIUM OF THE ECTODOMAIN OF SIV. GP41: INSIGHT INTO THE MECHANISM OF PEPTIDE INHIBITION OF HIV INFECTION,” Protein Sci. 8:1904-1907), K₂>>K₁. Taking K_{ref}^{trimer} as a reference point, the overall equilibrium association constant between monomeric and homotrimeric species of L is given by α K_{ref}^{trimer}, between monomeric and homotrimeric species of M by λK_{ref}^{trimer} (note that λ also subsumes any difference in the energetics of trimerization between the pre-hairpin intermediate in the membrane and the ectodomain of gp41 in free solution), and between monomeric species of L and M and heterotrimeric species of M and L by 3β K_{ref}^{trimer} (where the factor 3 is a statistical factor). The scheme in FIG. 5, Panel a has three unknowns: α, β, and λM_{T}, where M_{T} is the total protein concentration (in monomer units). The data, however, are insufficient to determine all three parameters independently. Nonlinear least-squares fitting to the experimental data, optimizing the values of α and β, was carried out for values of λM_{T} ranging from 1.5×10⁻⁷ to 1.5×10⁻³ M (which corresponds to values of λ of 1.5×10⁴−1.5×10⁸ for M_{T}=10 pM, the probable concentration of protein in bulk solution, estimated on the basis of a concentration of 5×10³ cells/μl and ˜5000 gp41 trimers/cell). (Note that the concentrations of the various species in the scheme shown in FIG. 5, Panel a as a function of total ligand concentration, L_{T}, were calculated numerically by integration of the differential equations describing the reactions to essentially infinite time.) The optimized values of α and β depend on the product λM_{T}, and the results are therefore equally valid for a wide range of M_{T} concentrations. The data can be equally well fitted for values of λM_{T} ranging from 10⁻⁷ to 10⁻³ M with α varying from ˜10 to 0.1 and β varying from 1 to 10 (FIG. 5, Panel b). Best fits to the experimental fusion inhibition data for λM_{T}=1.5×10⁻⁵ and 1.5×10⁻⁴ M are shown in FIG. 5, Panel c; the optimized values of α are 1.07 and 0.34 (with error estimates of ˜40%), respectively, and of β are 2.97 and 5.76 (with error estimates of 10%), respectively. The resulting curves are essentially indistinguishable from each other as well as from that obtained with a Langmuir isotherm. The occupancy of the various species relative to M_{T} and L_{T} are shown in FIG. 5, Panels d and e, respectively. For this set of parameters, the fraction M in the trimeric state in the absence of ligand is ˜86% for λM_{T}=1.5×10⁻⁵ M and ˜97% for λM_{T}=1.5×10⁻⁴M; the value of L_{T} at which 50% of L is monomeric is ˜2×10⁻⁶ and 5×10⁻⁶ M, respectively. The occupancy of homodimeric ligand is less than 1% of L_{T}; likewise the occupancy of homodimeric (MM) and heterodimeric (LM) protein is less than 1% of M_{T} for all values of L_{T}. Both MML and MLL heterotrimers are formed with the MML heterotrimer peaking at concentrations of L_{T} slightly less than that at which 50% of the ligand is monomeric.

The above calculations reveal two important findings. First, despite the complexities introduced by multiple homo- and hetero-oligomerization states, which might lead one to predict a complex relationship between fusion and total ligand concentration, a scheme such as that depicted in FIG. 5, Panel a can still yield rather simple inhibition data that is readily characterized by a Langmuir isotherm. Second, the values for the various equilibrium constants for trimerization required to best fit the experimental fusion data are entirely compatible with the experimentally measured value for the equilibrium constant for trimerization of the ectodomain of HIV-1 gp41 in solution.

In the best fit calculations described above and depicted in FIG. 5, only the homotrimeric form of the pre-hairpin intermediate of gp41, MMM, is considered to be fusion-active. If the calculations are repeated assuming that the heterotrimer, MML, containing only one molecule of N36^{Mut(e,g)}, is also fusion-active, the resulting theoretical curves do not reproduce the experimental data. One can therefore conclude that the energetics of formation of a five-helix bundle comprising a heterotrimeric internal coiled-coil consisting of two N-helices of gp41 and one N36^{Mut(e,g)} helix surrounded by two C-helices of gp41 is not sufficiently favorable to bring the target and viral membranes into sufficiently close proximity for fusion to take-place.

### EXAMPLE 5

### Engineering Of Peptides Derived From The N-Helix Of The Ectodomain Of gp41

Using rational design, two peptides have been engineered derived from the N-helix of the ectodomain of gp41. The parent peptide, N36, corresponds to residues 546-581 of HIV-1 Env and encompasses the N-terminal helix of gp41. The N36^{Mut(a,d)} peptide was designed to remove interactions leading to self-association and the formation of a trimeric coiled-coil of N-helices while preserving those residues that interact with the C-helix of the ectodomain of gp41. The absence of any fusion-inhibitory activity of N36^{Mut(a,d)} leads one to conclude that the C-region of gp41 can only interact with a trimeric coiled-coil of N-helices. The N36^{Mut(e,g)} peptide was designed to preserve the interactions leading to self-association while replacing those residues that interact with the C-region. N36^{Mut(e,g)} forms a monodisperse trimer in solution that does not interact with the C-region of gp41 and yet still inhibits fusion about 50-fold more effectively than the native gp41 sequence (i.e. N36) from which it was derived. These results can only be explained by the existence of a dynamic equilibrium between monomeric and trimeric coiled-coil forms of the N-region of gp41 in the pre-hairpin intermediate on a time scale sufficiently fast to permit subunit exchange and the consequent formation of heterotrimers of the N-helices of gp41 and N36^{Mut(e,g)}. Thus, N36^{Mut(e,g)} disrupts the homotrimeric coiled-coil of N-helices in the pre-hairpin intermediate state of gp41 and represents a novel third class of gp41-targeted fusion inhibitor. The other two classes of inhibitors bind to either the homotrimeric coiled-coil of N-helices (e.g. C34 and 120) or to the exposed C-region (e.g. N_{CCG}-gp41 and 5-helix) of gp41 in the pre-hairpin intermediate state. Since C34 (and presumably T20) also binds to N_{CCG}-gp41 and 5-helix (Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489; Root, M. J. et al. (2001) “PROTEIN DESIGN OF AN HIV-1 ENTRY INHIBITOR,” Science 291:884-888), these two classes of inhibitors antagonize each other. In contrast, one would predict that the N36^{Mut(e,g)} class of inhibitors should act either additively or synergistically with either of the other two classes. Therefore, N36^{Mut(e,g)} may represent a promising lead for the design of clinically effective, novel fusion inhibitors.

Reference citations in the above specification refer to the following references:
(1) Freed, E. O. et al. (1995) “THE ROLE OF HUMAN IMMUNODEFICIENCY VIRUS TYPE 1 ENVELOPE GLYCOPROTEINS IN VIRUS INFECTION,” J. Biol. Chem. 270:23883-23886
(2) Eckert, D. M. et al. (2001) “MECHANISMS OF VIRAL MEMBRANE FUSION AND ITS INHIBITION,” Annu. Rev. Biochem. 70:777-810
(3) Moore, J. P. et al. (1997) “CO-RECEPTORS FOR HIV-1 ENTRY,” Curr. Opin. Immunol. 9:551-562
(4) Caffrey, M. et al. (1998) “THREE-DIMENSIONAL SOLUTION STRUCTURE OF THE 44 KDA ECTODOMAIN OF SIV GP41,” EMBO J. 17:4572-4584
(5) Chan, D. C. et al. (1997) “CORE STRUCTURE OF GP41 FROM THE HIV ENVELOPE GLYCOPROTEIN,” Cell 89:263-273
(6) Weissenhorn, W. et al. (1997) “ATOMIC STRUCTURE OF THE ECTODOMAIN FROM HIV-1 GP41,” Nature 387:426-430
(7) Tan, K. J. et al. (1997) “ATOMIC STRUCTURE OF A THERMOSTABLE SUBDOMAIN OF HIV-1 GP41,” Proc. Natl. Acad. Sci. (U.S.A.) 94:12303-12308.
(8) Malashkevich, V. N. et al. (1998) “CRYSTAL STRUCTURE OF THE SIMIAN IMMUNODEFICIENCY VIRUS (SIV) GP41 CORE: CONSERVED HELICAL INTERACTIONS UNDERLIE THE BROAD INHIBITORY ACTIVITY OF GP41 PEPTIDES,” Proc. Natl. Acad. Sci. U.S.A. 95, 9134-9139
(9) Wild, C. T., et al. (1992) “A SYNTHETIC PEPTIDE INHIBITOR OF HUMAN IMMUNODEFICIENCY VIRUS REPLICATION: CORRELATION BETWEEN SOLUTION STRUCTURE AND VIRAL INHIBITION,” Proc. Natl. Acad. Sci. U.S.A. 89:10537-10541.
(10) Wild,. C. T. et al. (1994) “PEPTIDES CORRESPONDING TO A PREDICTIVE ALPHA-HELICAL DOMAIN OF HUMAN IMMUNODEFICIENCY VIRUS TYPE 1 GP41 ARE POTENT INHIBITORS OF VIRUS INFECTION,” Proc. Natl. Acad. Sci. U.S.A. 91:9770-9774
(11) Chan, D. C. et al. (1998) “EVIDENCE THAT A PROMINENT CAVITY IN THE COILED COIL OF HIV TYPE 1 GP41 Is AN ATTRACTIVE DRUG TARGET,” Proc. Natl. Acad. Sci. U.S.A. 95:15613-15617
(12) Eckert, D. M. et al. (2001) “DESIGN OF POTENT INHIBITORS OF HIV-1 ENTRY FROM THE GP41 N-PEPTIDE REGION,” Proc. Natl. Acad. Sci. U.S.A. 98:11187-11192
(13) Furuta, R. A. et al. (1998) “CAPTURE OF AN EARLY FUSION-ACTIVE CONFORMATION OF HIV-1 GP41,” Nat. Struct. Biol. 5:276-279
(14) Chan, D. C. et al. (1998) “HIV ENTRY AND ITS INHIBITION,” Cell 93:681 -684
(15) Kilby, J. M. et al. (1998) “POTENT SUPPRESSION OF HIV-1 REPLICATION IN HUMANS BY T-20, A PEPTIDE INHIBITOR OF G41-MEDIATED VIRUS ENTRY,” Nat. Med. 4:1302-1307
(16) Pozniak, A. (2001) “HIV FUSION INHIBITORS,” J. HIV Ther. 6:2-94
(17) Weissenhorn, W. et al. (1999) “STRUCTURAL BASIS FOR MEMBRANE FUSION BY ENVELOPED VIRUSES,” Mol. Membr. Biol. 16:3-9
(18) Kliger, Y. et al. (2000) “INHIBITION OF HIV-1 ENTRY BEFORE GP41 FOLDS INTO ITS FUSION-ACTIVE CONFORMATION,” J. Mol. Biol. 295:163-168
(19) Louis, J. M. et al. (2001) “DESIGN AND PROPERTIES OF N(CCG)-GP41, A CHIMERIC GP41 MOLECULE WITH NANOMOLAR HIV FUSION INHIBITORY ACTIVITY,” J. Biol. Chem. 276:29485-29489
(20) Root, M. J. et al. (2001) “PROTEIN DESIGN OF AN HIV-1 ENTRY INHIBITOR,” Science 291:884-888
(21) Wingfield, P. T. et al. (1997) “THE EXTRACELLULAR DOMAIN OF IMMUNODEFICIENCY VIRUS GP41 PROTEIN: EXPRESSION IN ESCHERICHIA COLI, PURIFICATION, AND CRYSTALLIZATION,” Protein Sci. 6, 1653-1660
(22) Caffrey, M. et al .(1999) “MONOMER-TRIMER EQUILIBRIUM OF THE ECTODOMAIN OF SIV GP41: INSIGHT INTO THE MECHANISM OF PEPTIDE INHIBITION OF HIV INFECTION,” Protein Sci. 8:1904-1907
(23) Peisajovich, S. G. et al. (2001) “SIV GP41 BINDS TO MEMBRANES BOTH IN THE MONOMERIC AND TRIMERIC STATES: CONSEQUENCES FOR THE NEUROPATHOLOGY AND INHIBITION OF HIV INFECTION,” J. Mol. Biol. 311:249-254
(24) Bohm, G. et al. (1992) “QUANTITATIVE ANALYSIS OF PROTEIN FAR UV CIRCULAR DICHROISM SPECTRA BY NEURAL NETWORKS,” Protein Eng. 5:191-195
(25) Perkins, S. J. (1986) “PROTEIN VOLUMES AND HYDRATION EFFECTS. THE CALCULATIONS OF PARTIAL SPECIFIC VOLUMES, NEUTRON SCATTERING MATCHPOINTS AND 280-NM ABSORPTION COEFFICIENTS FOR PROTEINS AND GLYCOPROTEINS FROM AMINO ACID SEQUENCES,” Eur. J. Biochem. 157:169-180
(26) Salzwedel, K. et al. (2000) “SEQUENTIAL CD4-CORECEPTOR INTERACTIONS IN HUMAN IMMUNODEFICIENCY VIRUS TYPE 1 ENV FUNCTION: SOLUBLE CD4 ACTIVATES ENV FOR CORECEPTOR-DEPENDENT FUSION AND REVEALS BLOCKING ACTIVITIES OF ANTIBODIES AGAINST CRYPTIC CONSERVED EPITOPES ON GP120,” J. Virol. 74:326-333
(27) Chance, E. M. et al. (1979) FACSIMILE, Atomic Energy Research Establishment Report R8775, Harwell, H. M. Stationary Office, London, UK
(28) Clore, G. M. (1983) In: COMPUTING IN BIOLOGICAL SCIENCE (Geisow, M. J., and Barrett, A. N., eds), pp. 313-348, Elsevier/North-Holland, N.Y.
(29) Lu, M. et al. (1995) “A TRIMERIC STRUCTURAL DOMAIN OF THE HIV-1 TRANSMEMBRANE GLYCOPROTEIN,” Nat. Struct. Biol. 2:1075-1082
(30) Jones, P. L. et al. (1998) “CONFORMATIONAL CHANGES IN CELL SURFACE HIV-1 ENVELOPE GLYCOPROTEINS ARE TRIGGERED BY COOPERATION BETWEEN CELL SURFACE CD4 AND CO-RECEPTORS,” J. Biol. Chem. 273:404-409
(31) Muñoz-Barroso, I. et al. (1998) “DILATION OF THE HUMAN IMMUNODEFICIENCY VIRUS-1 ENVELOPE GLYCOPROTEIN FUSION PORE REVEALED BY THE INHIBITORY ACTION OF A SYNTHETIC PEPTIDE FROM GP41,” J. Cell Biol. 140, 315-323

All publications and patents mentioned in this specification are herein incorporated by reference to the same extent as if each individual publication or patent application was specifically and individually indicated to be incorporated by reference. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A compound, comprising an N36 peptide variant or a derivative or pharmaceutically acceptable salt thereof, wherein said compound inhibits the fusion of HIV-1 to a human cell.

2. The compound of claim 1, wherein:
(A) the identities of the amino acid residues occupying the “e” and “g” positions of said N36 peptide variant are at most 89% identical to the amino acid residues occupying the “e” and “g” positions of N36 (SEQ ID NO:1), and
(B) the identities of the amino acid residues occupying the “non-e” and “non-g” positions of the N36 peptide variant are at least 70% identical to the amino acid residues occupying their counterpart positions in the N36 peptide;
wherein said N36 peptide variant possesses the ability to disrupt the internal trimeric coiled-coil of N-helices of gp41.

3. The compound of claim 1, wherein said N36 peptide variant differs in amino acid sequence from the sequence of N36 peptide (SEQ ID NO:1), by at least a substitution of an amino acid residue selected from the group consisting of: V₄, Q₆, L₁₁, A₁₃, Q₁₈, L₂₀, V₂₅, G₂₇ and Q₃₂, wherein the letters V, Q, L, A and G denote Valine, Glutamine, Leucine, Alanine and Glycine, respectively, and the number denotes the position of the residue in SEQ ID NO:1.

4. The compound of claim 3, wherein said N36 peptide variant differs in amino acid sequence from SEQ ID NO:1, by at least a substitution of at least two of said amino acid residues V₄, Q₆, L₁₁, A₁₃, Q₁₈, L₂₀, V₂₅, G₂₇ and Q₃₂.

5. The compound of claim 4, wherein said N36 peptide variant differs in amino acid sequence from SEQ ID NO:1, by at least a substitution of at least four of said amino acid residues V₄, Q₆, L₁₁, A₁₃, Q₁₈, L₂₀, V₂₅, G₂₇ and Q₃₂.

6. The compound of claim 5, wherein said N36 peptide variant differs in amino acid sequence from SEQ ED NO:1, by at least a substitution of at least five of said amino acid residues V₄, Q₆, L₁₁, A₁₃, Q₁₈, L₂₀, V₂₅, G₂₇ and Q₃₂.

7. The compound of claim 6, wherein said N36 peptide variant differs in amino acid sequence from SEQ ID NO:1, by at least a substitution of said amino acid residues V₄, Q₆, L₁₁, A₁₃, Q₁₈, L₂₀, V₂₅, G₂₇ and Q₃₂.

8. The compound of claim 7, wherein said N36 peptide variant has the amino acid sequence of N36^{Mut(e,g)} (SEQ ID NO:3):
SGIDQEQNNL TRLIEAQIHE LQLTQWKIKQ LLARIL.

9. A pharmaceutical composition comprising a therapeutically effective amount of an N36 peptide variant or a derivative or pharmaceutically acceptable salt thereof, wherein said compound inhibits the fusion of HIV-1 to a human cell, in admixture with a pharmaceutically acceptable excipient.

10. The pharmaceutical composition of claim 9, wherein:
(A) the identities of the amino acid residues occupying the “e” and “g” positions of said N36 peptide variant are at most 89% identical to the amino acid residues occupying the “e” and “g” positions of N36 (SEQ ID NO:1), and
(B) the identities of the amino acid residues occupying the “non-e” and “non-g” positions of the N36 peptide variant are at least 70% identical to the amino acid residues occupying their counterpart positions in the N36 peptide;
wherein said N36 peptide variant possesses the ability to disrupt the internal trimeric coiled-coil of N-helices of gp41.

11. The pharmaceutical composition of claim 9, wherein said N36 peptide variant differs in amino acid sequence from the sequence of N36 peptide (SEQ ED NO:1), by at least a substitution of an amino acid residue selected from the group consisting of: V₄, Q₆, L₁₁, A₁₃, Q₁₈, L₂₀, V₂₅, G₂₇ and Q₃₂, wherein the letters V, Q, L, A and G denote Valine, Glutamine, Leucine, Alanine and Glycine, respectively, and the number denotes the position of the residue in SEQ ID NO:1.

12. The pharmaceutical composition of claim 11, wherein said N36 peptide variant differs in amino acid sequence from SEQ ID NO:1, by at least a substitution of at least two of said amino acid residues V₄, Q₆, L₁₁, A₁₃, Q₁₈, L₂₀, V₂₅, G₂₇ and Q₃₂.

13. The pharmaceutical composition of claim 12, wherein said N36 peptide variant differs in amino acid sequence from SEQ ID NO:1, by at least a substitution of at least four of said amino acid residues V₄, Q₆, L₁₁, A₁₃, Q₁₈, L₂₀, V₂₅, G₂₇ and Q₃₂.

14. The pharmaceutical-composition of claim 13, wherein said N36 peptide variant differs in amino acid sequence from SEQ ID NO:1, by at least a substitution of at least five of said amino acid residues V₄, Q₆, L₁₁, A₁₃, Q₁₈, L₂₀, V₂₅, G₂₇ and Q₃₂.

15. The pharmaceutical composition of claim 14, wherein said N36 peptide variant differs in amino acid sequence from SEQ ID NO:1, by at least a substitution of said amino acid residues V₄, Q₆, L₁₁, A₁₃, Q₁₈, L₂₀, V₂₅, G₂₇ and Q₃₂.

16. The pharmaceutical composition of claim 15, wherein said N36 peptide variant has the amino acid sequence of N36^{Mut(e,g)} (SEQ ID NO:3): SGIDQEQNNL TRLIEAQIHE LQLTQWKIKQ LLARIL.

17. The pharmaceutical composition of claim 9, wherein said composition additionally contains an HIV protease inhibitor, an HIV reverse transcriptase inhibitor, an HIV integrase inhibitor, or an HIV fusion inhibitor.

18. A method of treating HIV infection that comprises providing to a recipient a therapeutically effective or a prophylactically effective amount of a pharmaceutical composition comprising a therapeutically effective amount of an N36 peptide variant or a derivative or pharmaceutically acceptable salt thereof, wherein said compound inhibits the fusion of HIV-1 to a human cell, in admixture with a pharmaceutically acceptable excipient.

19. The method of claim 18, wherein said N36 peptide variant has the amino acid sequence of N36^{Mut(e,g)} (SEQ ID NO:3):
SGIDQEQNNL TRLIEAQIHE LQLTQWKIKQ LLARIL.

20. The method of claim 18, wherein said pharmaceutical composition additionally contains an HIV protease inhibitor, an HIV reverse transcriptase inhibitor, an HIV integrase inhibitor, or an HIV fusion inhibitor.


SEQUENCE LISTING (4 sequences)

SEQ ID NO: 1 - protein, 36 aa, Human immunodeficiency virus type 1
SGIVQQQNNLLRAIEAQQHLLQLTVWGIKQLQARIL

SEQ ID NO: 2 - protein, 34 aa, Human immunodeficiency virus type 1
WMEWDREINNYTSLIHSLIEESQNQQEKNEQELL

SEQ ID NO: 3 - protein, 36 aa, Artificial Sequence
  Description of Artificial Sequence Synthetic peptide
SGIDQEQNNLTRLIEAQIHELQLTQWKIKQLLARIL

SEQ ID NO: 4 - protein, 36 aa, Artificial Sequence
  Description of Artificial Sequence Synthetic peptide
SGIVQQLNNQLRAEEANQHLEQLSVWGSKQNQARRL